(19) United States
(12) Patent Application Publication
(10) Pub. No.: US 2025/0144186 A1
(43) Pub. Date: May 8, 2025

(54) NOVEL THERAPEUTIC USES OF GARDNERELLA ENDOLYSINS

(71) Applicant: BioNTech SE, Mainz (DE)

(72) Inventors: Lorenzo Corsini, Mainz (DE); Vera Oberbauer, Mainz (DE); Lenka Podpera Tisakova, Mainz (DE); Timo Schwebs, Mainz (DE); Rocio Berdaguer Tarodo, Mainz (DE); Ann-Katrin Kieninger, Mainz (DE)

(21) Appl. No.: 18/837,311

(22) PCT Filed: Feb. 10, 2023
(86) PCT No.: PCT/EP2023/053316

§ 371 Date: Aug. 9, 2024

(30) Foreign Application Priority Data
Feb. 11, 2022 (EP) 22156413.1

(51) Int. Cl. A61K 38/47; A61K 45/06; A61P 31/04

(52) U.S. Cl. CPC A61K 38/47; A61K 45/06; A61P 31/04

(57) ABSTRACT

The present invention relates to new therapeutic uses of species-selective phage endolysins, in particular in the treatment of bacterial vaginosis (BV), even more particularly in the treatment of patients suffering from BV who previously failed a treatment with antibiotics, and/or patients suffering from BV wherein the infective bacteria are resistant to a treatment with antibiotics. The present invention also relates to pharmaceutical compositions for uses of the invention and methods of treatment using the same.

The present invention relates to new therapeutic uses of species-selective phage endolysins, in particular in the treatment of bacterial vaginosis (BV), even more particularly in the treatment of patients suffering from BV who previously failed a treatment with antibiotics, and/or patients suffering from BV wherein the infective bacteria are resistant to a treatment with antibiotics. The present invention also relates to pharmaceutical compositions for uses of the invention and methods of treatment using the same.

Bacterial vaginosis (BV), also been referred to in the literature as bacterial vaginitis, non-specific vaginosis and non-specific vaginitis, is the most common vaginal infection worldwide and is associated with significant adverse consequences including preterm labor and delivery, post-partum endometritis and an increased risk of HIV acquisition. It is a dysbiosis of the vagina where the commensal Lactobacilli are displaced by a polymicrobial biofilm, the pH increases from the natural 3.5-4.5 up to 5.5, and a malodorous fluid forms. It is most commonly defined as a pathological state characterized by the loss of normal vagina flora, particularly of H₂O₂-producing species of Lactobacillus, and the simultaneous overgrowth of anaerobic bacteria including Gardnerella vaginalis (G. vaginalis). This organism, first called Haemophilus vaginalis and repeatedly renamed as more information about its characteristics became available, is now classified as G. vaginalis which, until 2018, was considered to be the sole member of the genus Gardnerella. However, in early 2019 it was shown that the genus Gardnerella actually contains at least 13 species, and the most frequent ones were renamed G. vaginalis sensu stricto, G. leopoldii, G. piotii, and G. swidsinskii (Vaneechoutte et al., 2019 Int. J. Syst. Evol. Biol. 898661). Bacteria of the genus Gardnerella are special in that they are Gram-variable, i.e. they do not form the outer membrane defining the Gram-negative species. The cell wall is generally very thin and has only 10% or less content of peptidoglycan, which is why the crystal violet dye used for Gram staining does not always yield the deep purple color typical for Gram-positive species. Rather, Gardnerella cells can appear both Gram positive and negative in a Gram staining. Phylogenetic analysis based on 16S rRNA places Gardnerella in the gram-positive family Bifidobacteriales.

During BV, the epithelial surface is covered with a dense collection of Gardnerella bacteria forming an adherent biofilm on the vaginal epithelium, in which other species can proliferate, resulting in a polymicrobial biofilm that is frequently recalcitrant to treatment. Biofilms are adherent communities of microorganisms held together by a polymeric matrix composed of polysaccharides, proteins and/or nucleic acids. The distinct gene expression pattern, as well as the physical structure of biofilms increases bacterial resistance to many negative stimuli including chemical disinfectants, pH extremes, host immune defenses and antibiotics.

The recommended first-line therapy for BV is antibiotic treatment, predominately with nitroimidazole antibiotics such as metronidazole (MDZ), Tinidazole (TDZ) and secnidazole. and/or with clindamycin (CLI). Metronidazole (MDZ) belongs to the group of nitroimidazoles, and only gains its full activity when it is metabolized into its hydroxy metabolite (MDZ-OH). Antibiotics are effective in quickly reducing BV symptoms, but are associated with a high recurrence rate of up to 60% within six months of treatment. In a clinical trial where patients with recurrent BV were treated with 0.75% MDZ vaginal gel over 16 weeks, the probability of lasting cure was 70% after 16 weeks (i.e. 30% of patients had symptoms at the end of the 16 week treatment period) and declined to 34% 12 weeks after the end of therapy, i.e. at week 28 (Sobel et al., 2006, Am. J. Obstet. Gynecol. 194, 1283-1289). Besides a possible re-infection from sexual partners, the persistence of a residual infection has been postulated as a reason for recurrence, potentially due to the formation of a biofilm that protects BV-causing bacteria from antimicrobial therapy. Another reason may be antibiotic resistance of BV pathogens. Antibiotic resistance occurs naturally, but misuse of antibiotics in humans and animals is accelerating the process. Existing treatments thus fail to effectively penetrate biofilms. Accordingly, upon cessation of antibiotics treatment, biofilms re-grow, resulting in recurrent symptomatic presentations. Furthermore, treatment with antibiotics wipes the vaginal microbiome, despite leaving some rests of viable biofilm, which opens this ecological niche for other pathogens, e.g. fungi. A frequent effect of BV treatments is therefore candidiasis. Therefore, treatment failure and recurrent disease are common problems with antibiotic treatment.

There is thus a need for new methods and compositions to treat patients suffering from BV, especially in patients for whom antibiotic treatment is counter-indicated and/or have a record of adverse side effects upon antibiotic treatment. There is also a need for the treatment of BV in individuals who previously failed a treatment with antibiotics and/or who suffer from a bacterial infection wherein the infective bacteria are resistant to antibiotics treatment.

Thus, the technical problem underlying the present invention is the provision of novel means and methods for the treatment of BV in challenging clinical situations, such as in patients identified herein above.

The technical problem is solved by provision of the embodiments characterized in the claims.

In particular, the invention provides a Gardnerella-specific endolysin, preferably a recombinant Gardnerella-specific endolysin, for use in treating a bacterial vaginosis, wherein the endolysin is to be administered to a patient who previously failed a treatment with antibiotics, in particular a nitroimidazole and/or Clindamycin. The invention further provides a Gardnerella-specific endolysin, preferably a recombinant Gardnerella-specific endolysin, for use in treating a bacterial vaginosis, wherein the endolysin is to be administered to a patient who suffers from a bacterial vaginosis wherein the infective bacteria are resistant to a treatment with antibiotics, in particular a nitroimidazole and/or Clindamycin.

Endolysins are promising alternatives to the current antibiotics, due to their ability to eradicate biofilms, their low propensity to the development of resistance, and their specificity to individual genera or species of bacteria. Natural and genetically engineered Gardnerella-specific endolysins has been described (WO 2020/225335 A1, Landlinger et al. (2021, Pathogens 10, 1-19), or WO 2020/229802 A1). WO 2020/225335 describes specific recombinant Gardnerella-specific endolysins (e.g., H2B10) for use in a method of treating a Gardnerella infection such as BV, wherein said bacterial vaginosis is caused by Gardnerella vaginalis sensu stricto, Gardnerella leopoldii, Gardnerella piotii and/or Gardnerella swidsinskii. Examples 6 and 7 of WO 2020/225335 demonstrate that the endolysin H2B10 (as a representative of recombinant Gardnerella-specific endolysins) is superior to the antibiotics Metronidazole and Clindamycin, particularly in terms of minimal inhibitory concentration (MIC) on the growth in suspension of the Gardnerella strains. The results of WO 2020/225335 therefore document that (recombinant) Gardnerella-specific endolysins are superior to antibiotics in the treatment of BV in a general manner. In other words, (recombinant) Gardnerella-specific endolysins have been shown to be generally more effective on the growth in suspension of the Gardnerella strains as can be derived from the generally lower MIC values of the endolysins across the different Gardnerella species in comparison to those of the antibiotics. Appended Example 1 also documents that these Gardnerella-specific endolysins inhibit the growth in suspension of the Gardnerella strains (cf. Tables 1 and 2). Although endolysins have been shown to be a promising alternative to antibiotics to treat BV, data showing the potential to treat patients who have a history of bacterial vaginosis (i.e., who already experienced symptoms of BV or suffering from recurrent BV) and who previously failed a treatment with antibiotics have not yet been provided. Likewise, data showing the potential of endolysins to treat patients who suffer from BV wherein the infective bacteria are resistant, preferably highly resistant, to a treatment with antibiotics (e.g. wherein the antibiotic resistance has been acquired after one or more, preferably three or more, failed treatments with antibiotics) have also not yet been provided.

The present invention is based on the surprising and unexpected finding that the superiority of the endolysins over the antibiotics is even more pronounced on biofilm-grown Gardnerella strains (as illustrated in appended Example 2. Indeed, the biofilm-grown strains are more resistant to the action of the antibiotics Metronidazole (MDZ) and Clindamycin (CLI), but the recombinant Gardnerella-specific endolysin PM-477 is able to destroy these biofilm-grown organisms. This is of clinical relevance since Gardnerella strains in patients suffering from BV are typically in a biofilm state. The present invention is further based on the surprising and unexpected finding that Gardnerella spp. quickly develop resistance to antibiotic treatment using the antibiotic Metronidazole (MDZ) as an illustrative example (as illustrated in the appended Example 3). It is thus believed that a similar fast resistance formation against MDZ treatment occurs in BV patients during a course of antibiotic treatment using said antibiotic, triggering a vicious circle of BV therapy failure, further courses of MDZ treatment and further increasing tolerance of bacteria to this antibiotic. Because it is surprisingly found herein that the resistance status across strains is comparable for the antibiotics Metronidazole (MDZ) and Tinidazole (TDZ) (appended Example 1), it thus believed that a similar fast resistance formation against TDZ treatment occurs in BV patients during a course of antibiotic treatment using said antibiotic. In addition, because MDZ and TDZ both belong to the class of the nitroimidazole antibiotics, i.e., a class of antibiotics that share similar chemical structures, it is thus believed that a similar fast resistance formation against the antibiotic treatment occurs in BV patients during a course of antibiotic treatment using any one of the antibiotics from the group consisting of the nitroimidazole antibiotics, including MDZ, TDZ and secnidazole. Further, it is surprisingly found herein that both antibiotics MDZ and Clindamycin (CLI) are ineffective on the majority of tested Gardnerella isolates if they are grown as biofilms (appended Example 2). Because Gardnerella strains in patients suffering from BV are typically in a biofilm state, it is thus believed that treatment with CLI would also result in BV therapy failure and that a similar fast resistance formation against CLI treatment occurs in BV patients during a course of antibiotic treatment using said antibiotic. The present invention is further based on the surprising and unexpected finding that, in contrast to said antibiotics treatment, a recombinant Gardnerella-specific endolysin (as illustrated with the endolysin “PM-477”, also referred to as “H2B10” interchangeably, the amino acid sequence of which is set forth in SEQ ID NO: 1) is highly active on Gardnerella preformed biofilms (appended Example 2), does not induce resistance formation (appended Example 3) and is fully active on antibiotics-resistant Gardnerella strains, particularly those Gardnerella strains that are highly resistant to antibiotics and/or that acquired antibiotics resistance after one or more (failed) antibiotics treatment (appended Example 4). Therefore, the present invention is based on the surprising and unexpected finding that a (recombinant) Gardnerella-specific endolysin is useful to treat BV, especially in patients with recurrent BV (i.e., who have a history of bacterial vaginosis, i.e. who already experienced symptoms of BV) and who failed treatment with antibiotics, in particular a nitroimidazole (e.g., MDZ) and/or CLI, and/or patients who suffers from a bacterial vaginosis wherein the infective bacteria are resistant to antibiotics treatment (e.g. wherein the antibiotic resistance has been acquired after one or more, preferably three or more, failed treatments with antibiotics), in particular a nitroimidazole (e.g., MDZ) and/or CLI.

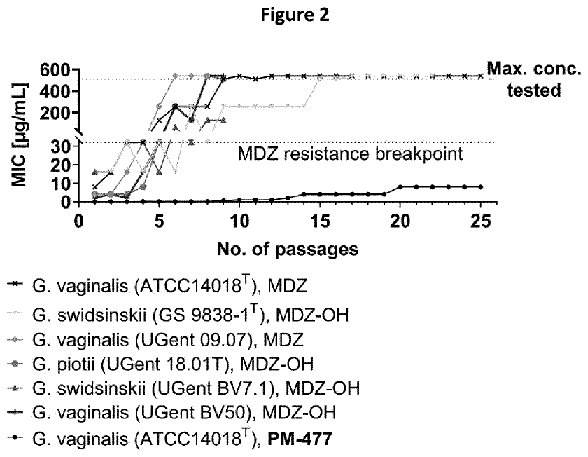

The present invention is to be clearly distinguished from the therapeutic uses described in WO 2020/225335 by the particular group of subjects to be treated. The new therapeutic use of the invention is specifically directed to patients who previously failed a treatment with antibiotics and/or who suffer from bacterial vaginosis wherein the infective bacteria are resistant to a treatment with antibiotics, e.g., patients suffering from BV caused by Gardnerella strains which acquired resistance to antibiotics after one or more failed treatment with said antibiotics. This new clinical situation is represented in the appended examples by “passaging” the Gardnerella strains with MDZ (as a representative of an antibiotic used to treat BV). In the context of the present invention “passaging” refers to treating bacterial strains with antibiotics in such a way that the growth is impaired but not completely inhibited. Therefore, this in-vitro setting intends to artificially reproduce the effects that a failed treatment with antibiotics will have on a patient suffering from a bacterial infection (e.g., BV). As it is shown in appended Example 3 and FIG. 2, the resistance of the Gardnerella strains to the antibiotic is greatly increased (as represented by increased MIC values) after each failed treatment (i.e. each “passage”) with antibiotics (e.g., MDZ). After a number of passaging rounds (e.g., 5 or more), the majority of the Gardnerella strains tested can no longer be inhibited even by the maximal concentration in use, meaning that these Gardnerella strains became highly resistant to antibiotics treatment (as defined herein below). Surprisingly and unexpectedly, the present inventors show herein that this deleterious effect is not seen upon treatment with recombinant Gardnerella-specific endolysins (as illustrated with the endolysin PM-477). Indeed, appended Example 3 and FIG. 2 show that the MIC of a recombinant Gardnerella-specific endolysin only slightly increased even after 25 rounds of passaging. Even more surprising and unexpecting, it is shown herein that recombinant Gardnerella-specific endolysins (as illustrated with the endolysin PM-477) are still effectively reducing the growth of the Gardnerella strains that acquired resistance to antibiotics (as illustrated with MDZ as a representative of an antibiotic used to treat BV), even the strains displaying the highest resistance. Therefore, it is shown herein that recombinant Gardnerella-specific endolysins are surprisingly suitable for use in treating a patient suffering from BV, wherein said patient previously failed a treatment with antibiotics and/or wherein the infective bacteria of said BV are (highly) resistant to a treatment with antibiotics, in particular wherein said antibiotics are a nitroimidazole and/or Clindamycin.

Accordingly, in a first aspect of the invention, the Gardnerella-specific endolysin, preferably recombinant Gardnerella-specific endolysin, as described herein is for use in treating a bacterial vaginosis, wherein the endolysin is to be administered to a patient who previously failed a treatment with antibiotics.

As used herein, “a patient who previously failed a treatment with antibiotics” refers to a patient who has a history of bacterial vaginosis, i.e. who contracted (or already experienced) symptoms of bacterial vaginosis in the past, who has been treated for said bacterial vaginosis by antibiotics and who relapsed, i.e. for whom the symptoms reappeared. Such a patient can also be referred to as a patient suffering from recurrent BV. As used herein, the terms “a patient who has history with bacterial vaginosis”, “a patient who already experienced symptoms of bacterial vaginosis” and “a patient suffering from recurrent BV” can be used interchangeably and include, but are not limited to, patients who had one or more episodes of BV, preferably two or more episodes of BV, more preferably two or more episodes of BV in 6 months, even more preferably two or more episodes of BV within the last 6 months, or preferably three or more episodes of BV, more preferably three or more episodes of BV in 12 months, even more preferably three or more episodes of BV within the last 12 months. Methods to diagnose BV in a patient or to prove episodes of BV are known to the skilled person in the art, e.g., BV can be diagnosed clinically by using clinical criteria (such as, e.g., the Amsel's diagnostic criteria) or microscopically, by determining the Nugent score from a vaginal Gram stain. Without being bound by any theory, relapses (or recurrences) of BV in patients after a treatment with antibiotics might be caused by the persistence of a residual infection due to the resistance of the infective bacteria to the antibiotics used. Accordingly, in one preferred embodiment of this first aspect of the invention, the patient suffers from a bacterial vaginosis wherein the infective bacteria are resistant to antibiotics treatment. Thus, in one preferred aspect of the invention, the Gardnerella-specific endolysin described herein is for use in treating a bacterial vaginosis, wherein the endolysin is to be administered to a patient who previously failed a treatment with antibiotics and who suffers from a bacterial vaginosis wherein the infective bacteria are resistant to antibiotics treatment. As defined herein below, the infective bacteria of the BV to be treated herein are even more preferably highly resistant to antibiotics treatment. In one preferred embodiment of the therapeutic uses of the invention, the patient to be treated suffers from BV wherein the infective bacteria of said BV are resistant, preferably highly resistant, to a treatment with Metronidazole, Tinidazole, Secnidazole, Clindamycin or any combination thereof. In one more preferred embodiment of the therapeutic uses of the invention, the patient to be treated suffers from BV wherein the infective bacteria of said BV are resistant, preferably highly resistant, to a treatment with Metronidazole and/or Clindamycin. In one even more preferred embodiment of the therapeutic uses of the invention, the patient to be treated suffers from BV wherein the infective bacteria of said BV are resistant, preferably highly resistant, to a treatment with Metronidazole.

In a second aspect of the invention, the Gardnerella-specific endolysin, preferably recombinant Gardnerella-specific endolysin, as described herein is for use in treating a bacterial vaginosis, wherein the endolysin is to be administered to a patient who suffers from a bacterial vaginosis wherein the infective bacteria are resistant to antibiotics treatment.

In the context of the present invention, “resistance” of bacterial strains (preferably Gardnerella strains) with regards to antibiotics refers to the ability of the strains to resist the activity of the antibiotic to which it was previously susceptible and permits them to survive the antibiotics treatment. Antibiotic resistance might occur naturally (“intrinsic resistance”) or might be induced by the misuse of antibiotics in humans and animals (“acquired resistance”). Methods how to determine resistance or susceptibility of bacterial strains to antibiotics are known to the skilled person. As an example, EUCAST breakpoints for Gram-positive anaerobes (v11, 2021) can be used. An alternative definition is given in Petrina et al. (2017, Anaerobe 47, 115-119), where slightly higher resistance breakpoints are used, because the topical formulations of some nitroimidazoles and Clindamycin can establish concentrations in the mg/ml range in vaginal fluid, much higher than what is achievable with orally delivered antibiotics. According to this alternative, more stringent definition (also used in the appended examples), Resistance (R) can be defined as a MIC value superior or equal to 32 μg/ml for Metronidazole and as a MIC value superior 8 μg/ml for Clindamycin, whereas Sensitivity (S) can be defined as a MIC value inferior or equal to 8 μg/ml for Metronidazole and inferior or equal to 2 μg/ml for Clindamycin. The terms “Minimum Inhibitory Concentration” and “MIC” are used herein interchangeably and refer to the lowest concentration of a chemical, usually a drug, which prevents visible growth of bacterium. MIC can be defined as the minimal concentration of antibiotic at which no growth was detectable after 48 h by OD measurement. Accordingly, in one preferred embodiment of the therapeutic uses of the invention, the patient to be treated suffers from BV wherein the infective bacteria are resistant to antibiotics treatment as defined by EUCAST breakpoints. Other generally accepted resistance criteria might be used instead of and/or besides the EUCAST definition or the alternative definition mentioned above (Petrina et al., 2017, Anaerobe 47, 115-119) to define the resistance of the infective bacteria to antibiotics treatment in the context of the present invention.

As will be known to the skilled person in the art, “resistance” and “susceptibility” might also be defined in terms of MBC and/or MBEC values. The terms “Minimum Bactericidal Concentration” or “MBC” refer to the lowest concentration of an antibacterial agent required to kill a particular bacterium. Usually, the MBC90 or MBC99.5 is measured, i.e. the antibiotic concentration killing 90% or 99.5%, respectively, of cells within a defined time. MBC can be defined as the minimal concentration fully eradicating a suspension of, e.g., 2.5×10⁷ CFU/ml. While MIC is the lowest concentration of an antibacterial agent necessary to inhibit visible growth, MBC is the minimum concentration of an antibacterial agent that results in bacterial death of all cells in suspension up to a defined limit of detection, resulting in killing of at least 90% (MBC90) or at least 99.5% (MBC99.5) of bacteria. The terms “Minimum Biofilm Eradicating Concentrations” or “MBEC” refer to the lowest concentration of an antibacterial agent required to reduce a population of bacteria growing as biofilm below the limit of detection. The details of the method used in this disclosure to measure MBEC are described in the Materials and Methods section of the Examples.

Without being bound by any theory, the resistance of the infective bacteria to antibiotics might be involved in (or being partially or substantially responsible of) the relapses (or recurrences) of BV in patients suffering from recurrent BV. Therefore, a patient suffering from bacterial vaginosis wherein the infective bacteria are resistant to a treatment with antibiotics is likely to fail a treatment with antibiotics. Accordingly, in one preferred embodiment of this second aspect, the patient to be treated and who suffers from bacterial vaginosis wherein the infective bacteria are resistant to a treatment with antibiotics is a patient who is prone to fail a treatment with antibiotics. As used herein, a patient “who is prone to fail a treatment with antibiotics” refers to a patient who is at high risk of failing a treatment with antibiotics, i.e. for whom the likelihood of relapse (e.g., within 12 months) is very high if said patient were to be treated with antibiotics. Methods to determine whether a patient will be prone to fail a treatment with antibiotics are known to the skilled person. As an example, a clinical sample can be collected and the resistance of the vaginal microflora strains assessed. Likewise, a patient suffering from bacterial vaginosis wherein the infective bacteria are resistant to a treatment with antibiotics is likely to have already (i.e. previously) failed a treatment with antibiotics (as defined above). Accordingly, in one further preferred embodiment of this second aspect, the patient to be treated and who suffers from bacterial vaginosis wherein the infective bacteria are resistant to a treatment with antibiotics is a patient who previously failed a treatment with antibiotics.

As it is surprisingly shown in the appended examples, while Gardnerella-strains acquired more and more antibiotics resistance after each “passaging” with said antibiotics, all strains remain susceptible to the Gardnerella-specific endolysins (see appended Example 4). Indeed, as shown in Table 4 below, before passaging the Gardnerella-strains display MIC values between 8 and 256 μg/mL for MDZ, whereas the MIC values for the corresponding passaged strains are above 252 μg/mL or 2048 μg/mL, meaning that the antibiotics resistance of the Gardnerella-strains increased after each passaging, up to the point where the (passaged) Gardnerella-strains can tolerate very high concentration of antibiotics without any loss of viability. After passaging, the (passaged) Gardnerella-strains thus became highly resistant to antibiotics treatment. According to the above definition of resistance, “High Resistance” (HR) can be defined as a MIC value superior or equal to 256 μg/ml for Metronidazole and a MIC value superior to 64 μg/ml for Clindamycin, i.e. 8-fold higher than the resistance breakpoints of the alternative definition, which are themselves already higher than the standard resistance breakpoints of the EUCAST definition. In contrast to antibiotics, there is no resistance breakpoints for the Gardnerella-specific endolysins as described herein (as illustrated with PM-477) and even said “passaged” or “highly resistant” Gardnerella-strains remain susceptible to the treatment with said endolysins. Accordingly, in one embodiment of the therapeutic uses of the invention, the patient to be treated suffers from BV wherein the infective bacteria of said BV are resistant, preferably highly resistant, to antibiotics treatment, preferably as defined by EUCAST breakpoints. In one preferred embodiment of the therapeutic uses of the invention, the patient to be treated suffers from BV wherein the infective bacteria of said BV are resistant, preferably highly resistant, to a treatment with Metronidazole, Tinidazole, Secnidazole, Clindamycin or any combination thereof. In one more preferred embodiment of the therapeutic uses of the invention, the patient to be treated suffers from BV wherein the infective bacteria of said BV are resistant, preferably highly resistant, to a treatment with Metronidazole and/or Clindamycin. In one even more preferred embodiment of the therapeutic uses of the invention, the patient to be treated suffers from BV wherein the infective bacteria of said BV are resistant, preferably highly resistant, to a treatment with Metronidazole.

In another aspect of the invention, the Gardnerella-specific endolysin, preferably recombinant Gardnerella-specific endolysin, described herein is for use in treating a patient suffering from bacterial vaginosis, wherein said patient previously failed a treatment with antibiotics and/or wherein the infective bacteria of said bacterial vaginosis are resistant to antibiotics treatment.

All of the herein-mentioned aspects of the invention are encompassed by “the therapeutic uses of the invention”.

As used herein, the terms “treatment with antibiotics” and “antibiotics treatment” are used interchangeably and preferably refer to a treatment with the antibiotics recommended or approved for the treatment of BV. Antibiotics that are currently recommended or approved for the treatment of BV include the Nitroimidazoles, including but not limited to Metronidazole, Tinidazole, and Secnidazole, and the Clindamycin. Thus, in one embodiment, said “treatment with antibiotics” or “antibiotics treatment” is a treatment with a nitroimidazole and/or Clindamycin. In a preferred embodiment, the treatment with antibiotics described herein is a treatment with Metronidazole, Tinidazole, Secnidazole, Clindamycin or any combination thereof. In a more preferred embodiment, the antibiotics treatment described herein is a treatment with Metronidazole, and/or Clindamycin. In an even more preferred embodiment, the antibiotics treatment described herein is a treatment with Metronidazole.

In one embodiment of the therapeutic uses of the invention, the patient to be treated suffers from recurrent bacterial vaginosis (as defined herein above). In one embodiment of the therapeutic uses of the invention, the patient to be treated has history with bacterial vaginosis. In one embodiment of the therapeutic uses of the invention, the patient to be treated already experienced symptoms of bacterial vaginosis. In one embodiment of the therapeutic uses of the invention, the patient to be treated had one or more, preferably two or more episodes of BV, more preferably two or more episodes of BV in 6 months, even more preferably two or more episodes of BV within the last 6 months, or preferably three or more episodes of BV, more preferably three or more episodes of BV in 12 months, even more preferably three or more episodes of BV within the last 12 months. Thus, in one embodiment, the Gardnerella-specific endolysin, preferably recombinant Gardnerella-specific endolysin, described herein is for use in treating a bacterial vaginosis, wherein said bacterial vaginosis is recurrent bacterial vaginosis. In a preferred embodiment, the Gardnerella-specific endolysin, preferably recombinant Gardnerella-specific endolysin described herein, is for use in treating a bacterial vaginosis, wherein the endolysin is to be administered to a patient who had two or more episodes of BV in 6 months or who had three or more episodes of BV in 12 months. In another preferred embodiment, the Gardnerella-specific endolysin, preferably recombinant Gardnerella-specific endolysin, described herein is for use in treating a patient suffering from a bacterial vaginosis, wherein said patient had two or more episodes of BV in 6 months or had three or more episodes of BV in 12 months. In another preferred embodiment, the Gardnerella-specific endolysin, preferably recombinant Gardnerella-specific endolysin, described herein is for use in treating a patient who has history with bacterial vaginosis.

As used herein “Bacterial Vaginosis” (BV), also been referred to in the literature as bacterial vaginitis, non-specific vaginosis and non-specific vaginitis refers to the most common vaginal infection worldwide. In one embodiment, BV is defined as a pathological state characterized by the loss of normal vagina flora, particularly of H₂O₂-producing species of Lactobacillus, and the simultaneous overgrowth of anaerobic bacteria, often including those from the genus Gardnerella. The genus Gardnerella contains at least 13 species, and the most frequent ones were renamed G. vaginalis sensu stricto, G. leopoldii, G. piotii, and G. swidsinskii (Vaneechoutte et al., 2019, Int. J. Syst. Evol. Microbiol. 69, 679-687). In one preferred embodiment of the therapeutic use of the invention, the BV to be treated is a bacterial infection characterized by the presence of least one strain of the Gardnerella genus selected from the group consisting of Gardnerella vaginalis sensu strict, Gardnerella leopoldii, Gardnerella piotii and Gardnerella swidsinskii, and any other Gardnerella species. Thus, in one embodiment, the Gardnerella-specific endolysin, preferably recombinant Gardnerella-specific endolysin, described herein is for use in treating a bacterial vaginosis, wherein said bacterial vaginosis is characterized by the presence of infective bacteria of species Gardnerella vaginalis sensu stricto, Gardnerella leopoldii, Gardnerella piotii, Gardnerella swidsinskii, and/or any other species in the genus Gardnerella. It is understood herein that a bacterial vaginosis which “is characterized by the presence of” (or “is caused by” as used herein interchangeably) certain bacterial species in the genus Gardnerella refers to the overgrowth of said bacteria (also referred herein as “infective bacteria”) in the vagina microflora of a patient, leading to vaginal dysbiosis and/or the loss of Lactobacillus dominance. Methods to determine whether a bacterial vaginosis is characterized by the presence of infective bacteria of bacterial species in the genus Gardnerella are known to the skilled person. As an example, a PCR test that checks for presence of Gardnerella strains to diagnose BV can be used.

In one embodiment of the therapeutic uses of the invention, the BV to be treated by the Gardnerella-specific endolysin described herein is characterized by the presence of a Gardnerella strain (i.e. a strain from the genus Gardnerella) which is resistant to one or more antibiotics. In one preferred embodiment, said one or more antibiotic(s) is/are selected from the group consisting of the nitroimidazoles and Clindamycin. In one even more preferred embodiment, said Gardnerella strain is resistant to Metronidazole, Tinidazole, Secnidazole, Clindamycin or any combination thereof. In one even more preferred embodiment, said Gardnerella strain is resistant to Metronidazole and/or Clindamycin. In one even more preferred embodiment, the said Gardnerella strain is resistant to Metronidazole. In one most preferred embodiment, said Gardnerella strain is resistant or highly resistant to Metronidazole.

The term “endolysin” as used herein refers to a polypeptide usually produced by bacteriophages to digest the host bacteria cell wall and release bacteriophage progeny. An endolysin is a cell-wall lytic enzyme encoded by bacteriophages which have the ability to hydrolyze the cell-wall of target bacteria when added exogenously (lysis-from-without). This novel class of antibacterials has important advantages over classical antibiotics, e.g. a novel mode of action; a narrow spectrum of susceptible bacteria; rapid killing of both stationary- and exponentially-growing bacteria; activity on mucous membranes and bacterial biofilms; low probability of developing resistances; and reduced impact on normal microbiota. These unique features have boosted the interest on the biotechnological and pharmacological exploitation of lysins and their recent inclusion among the top current alternatives to fight antibiotic resistances. Endolysins often consist of two or more domains: at least one catalytic domain, such as a hydrolase domain (typically located at the N-terminal of the polypeptide), which cleaves specific motifs in the peptidoglycan layer, and often one or more cell wall binding domains (classically located at the C-terminal of the polypeptide), which is involved in the specific binding and processing of the bacterial peptidoglycan. Although providing a general organization for endolysin structure, this typical architecture is not a defined characteristic of all endolysins. Endolysins from Gram-positive bacteria and their phages usually comprise at least one catalytic domain and one or more cell wall-binding domains. In contrast, many endolysins produced by Gram-negative species or their phages only contain the catalytic domain, though modular endolysins have also been reported. The catalytic units dictate the type of peptidoglycan (PG) bond to be cleaved, whereas the cell wall-binding domain(s) largely determines the lytic spectrum by specific recognition of cell wall elements distributed in genus-, or species/strain-specific manner.

The endolysin to be used in the therapeutic uses of the present invention is preferably a recombinant endolysin which is Gardnerella-genus specific, i.e. it specifically targets bacteria that belong to the genus Gardnerella. The endolysin to be used in the therapeutic uses of the present invention further preferably has killing activity against species in the genus Gardnerella. For example, the endolysin to be used in the therapeutic uses of the present invention may have killing activity against Gardnerella vaginalis sensu stricto, Gardnerella leopoldii, Gardnerella piotii and/or Gardnerella swidsinskii, preferably against all of them. The killing activity of the endolysin to be used in the therapeutic uses of the present invention against Gardnerella is more preferably a genus-selective killing activity against Gardnerella. Herein “genus-selective killing activity” or “genus-specific bacteriolytic effect” means that the endolysin to be used in the therapeutic uses of the present invention does not have killing activity or bacteriolytic effect against bacteria in general. In particular, the endolysin to be used in the therapeutic uses of the present invention does not have killing activity against bacteria other than Gardnerella spp. Preferably, the endolysin to be used in the therapeutic uses of the present invention has genus-selective killing activity against Gardnerella, but not against Lactobacilli. In particular, it is preferred that said endolysin has no killing activity against Lactobacilli crispatus, Lactobacilli gasseri, and/or Lactobacilli jensenii. More preferably, said endolysin has no killing activity against all of these Lactobacilli, i.e. Lactobacilli crispatus, Lactobacilli gasseri, and Lactobacilli jensenii.

As used herein, the “killing activity” of an endolysin against particular bacteria can be defined as a reduction in the number of viable bacteria cells caused by the lysing activity of said endolysin. The killing activity of the endolysin against said bacteria can be complete meaning that 100% of the bacterial cells have been lysed or partial meaning that at least about 80%, at least about 90%, at least about 95%, at least about 99%, or at least about 99.9% of the bacterial cells have been lysed. The killing activity of an endolysin on a particular microorganism may be determined by standard procedures in the field including those based on the determination of the Minimum Inhibitory Concentrations (MICs) of an antimicrobial agent defined as the lowest concentration of said antimicrobial agent that inhibits the visible growth of a microorganism after overnight incubation as described in Andrews, 2001, J Antimicrobial Chemotherapy, 48, Suppl. SI, 5-16 or in “Document M7-A7, Methods for dilution antimicrobial susceptibility tests for bacteria that grow aerobically; Approved standards, 7th Edition, January 2006, vol. 26, No. 2” published by Clinical and Laboratory Standards Institute. Another suitable method for determining the killing activity of an endolysin is described in the example section of WO 2020/225335 and consists in measuring a decrease in optical density at 610-620 nm of a bacterial cell suspension and/or a decrease in Colony Forming Units (CFU) per milliliter of a bacterial cell suspension after exposure to the endolysin to be tested. The decrease of the Optical Density measured at 610-620 nm of a suspension of bacteria, the susceptibility of which is to be tested, can be determined in an in vitro turbidity assay performed in presence of purified endolysin. According to another embodiment, in an in vitro turbidity test, an endolysin has killing activity against Gardnerella when said endolysin decreases the OD (610-620 nm) of a suspension of at least one strain of Gardnerella bacteria by more than 20%, more than 30%, more than 40%, more than 50%, more than 60%, more than 70%, more than 80%, more than 90%, or more than 95%.

The endolysin to be used in the therapeutic uses of the present invention further preferably has killing activity against antibiotic resistant Gardnerella strains or highly antibiotic resistant Gardnerella strains as defined above. In one preferred embodiment, the endolysin to be used in the therapeutic uses of the present invention has killing activity against Gardnerella strains which are resistant to one or more antibiotics selected from the group consisting of the nitroimidazoles and Clindamycin. In one more preferred embodiment, the endolysin to be used in the therapeutic uses of the present invention has killing activity against Gardnerella strains which are resistant to Metronidazole, Tinidazole, Secnidazole, Clindamycin or any combination thereof. In one even more preferred embodiment, the endolysin to be used in the therapeutic uses of the present invention has killing activity against Gardnerella strains which are resistant to Metronidazole, and/or, Clindamycin. In one even more preferred embodiment, the endolysin to be used in the therapeutic uses of the present invention has killing activity against Gardnerella strains which are resistant to Metronidazole. In one more preferred embodiment, the endolysin to be used in the therapeutic uses of the present invention has killing activity against Gardnerella strains which are highly resistant to said antibiotics mentioned above.

In the therapeutic uses of the present invention, the endolysins are to be administered orally (e.g., as a pill) or locally (e.g., as a topical gel, lotion or cream or as a pessary, i.e. a vaginal suppository). In one preferred aspect of the therapeutic uses of the invention, the endolysin is to be administered locally, i.e. locally into the vagina of a female subject and/or, in a male subject into or on the glans penis, prepuce or urethral entry. Herein the term “(administration) into or on the glans penis” also includes “(administration) into and on the glans penis”. In line with this, the term “(administration) into or on the glans penis, prepuce or urethral entry of a male subject” also includes “(administration) into and on the glans penis and on the prepuce and on the urethral entry of a male subject”. As illustrative examples, the endolysin for uses of the present invention might be formulated as topical gel, lotion or cream that is to be inserted into the vagina of female subject and/or that is to be inserted into and/or applied on the glans penis of a male subject. As another illustrative example, the endolysin for uses of the present invention might inserted in the vagina of female subject in the form of a pessary (vaginal suppository). As another illustrative example, the endolysin for uses of the present invention might also be applied on a condom before sexual relations.

The optimum pH at which the endolysin to be used in the therapeutic uses of the present invention exhibits a killing activity, preferably genus-selective killing activity, against Gardnerella is comprised between about 4 and 6, preferably a pH about 5. Therefore, in one preferred aspect of the therapeutic uses of the present invention, the endolysin is to be co-administered with a compound or composition which adjusts the pH of the vagina to 4.0-6.0, preferably to 4.5-5.5, more preferably to about 5. Suitable compounds or compositions which adjusts the pH of the vagina include but are not limited to phosphate, lactic acid (e.g. the natural acidification substance which Lactobacilli secrete to establish an acidic milieu) or other organic acids, e.g. carboxy-substituted polymers.

The endolysin to be used in the therapeutic uses of the present invention is preferably a functional polypeptide, wherein the function comprises specifically targeting bacteria from the Gardnerella genus, more preferably specially killing bacteria from the Gardnerella genus. The endolysin to be used in the therapeutic uses of the present invention further preferably comprises a catalytic domain or a functional fragment thereof and/or a cell wall binding domain or a functional fragment thereof. The endolysin to be used in the therapeutic uses of the present invention might be a natural or a recombinant endolysin. The endolysin to be used in the therapeutic uses of the present invention most preferably a recombinant endolysin. The endolysin to be used in the therapeutic uses of the present invention is more preferably a recombinant endolysin comprising or consisting of
(i) a N-terminal catalytic domain, or a functional variant thereof;
(ii) a C-terminal cell-wall binding region, or a functional variant thereof, wherein the C-terminal cell-wall binding region comprises or consists of at least one cell-wall binding domain; and
(iii) optionally a linker region between the N-terminal catalytic domain and the C-terminal cell-wall binding region,
and has preferably a killing activity, more preferably genus-selective killing activity, against Gardnerella cells/strains.

In the context of the present disclosure, the term “recombinant endolysin” preferably refers to an endolysin which has been domain-swapped, as defined in WO 2020/225335. In line with this definition, the person skilled in the art readily understands that the “domain-swapped” or “recombinant” endolysins as described herein are non-naturally occurring endolysins. That is, the recombinant endolysin for use of the present invention has been modified by hand of man and excludes, by definition, natural endolysins, i.e. as it can be naturally found in nature. The appended examples as well as the teaching of WO 2020/225335 provide suitable method(s) how to generate the artificial endolysin of the invention.

The term “catalytic domain” or “enzymatic domain” refer to the part of the protein chain which contains the region where the catalyzed chemical reaction takes place. The “catalytic domain” as used herein refers to a functional polypeptide, wherein the function comprises the ability to lyse the cell wall of Gardnerella. In particular, the catalytic domain as a described herein can preferably modify and/or cleave a substrate in Gardnerella cell walls, preferably peptidoglycan. Preferably, the catalytic domain cleaves peptidoglycan in Gardnerella cell walls and can cause Gardnerella cell lysis. Preferably, the catalytic domain can modify and/or cleave bonds that are present in the cell wall and/or peptidoglycan of Gardnerella spp., such as Gardnerella vaginalis sensu stricto, Gardnerella leopoldii, Gardnerella piotii and/or Gardnerella swidsinskii, preferably all of them. Suitably, the catalytic domain does not modify and/or cleave a substrate, preferably peptidoglycan, present in cell wall of bacteria other than Gardnerella spp., preferably healthy vaginal commensal bacteria, such as Lactobacillus spp. including Lactobacilli crispatus, Lactobacilli gasseri, and/or Lactobacilli jensenii. The catalytic domain may be a N-acetylmuramidase, N-acetylmuramoyl-L-alanine amidases, L-alanoyl-D-glutamate endopeptidases, interpeptide bridge endopeptidases or N-acetyl-beta-D-glucosaminidases. Preferably, the N-terminal catalytic domain is a N-acetylmuramidase, most preferably a 1,4-beta-N-acetylmuramidase. The catalytic domain is preferably located N-terminally within the (recombinant) endolysin, thereby referred to as “N-terminal catalytic domain”, even more preferably the N-terminal catalytic domain is located N-terminally from the C-terminal cell-wall binding region within the (recombinant) endolysin.

The endolysin to be used in the therapeutic uses of the present invention preferably comprises a catalytic domain consisting of a polypeptide comprising or consisting of the amino acid sequence of any one of SEQ ID NOs: 2 to 10 or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOS: 2 to 10. As shown in WO 2020/225335, the most active catalytic domain is “H2” (SEQ ID NO: 3). Accordingly, in a preferred aspect of the present invention the catalytic domain is consisting of a polypeptide which comprises or consists of the amino acid sequence of SEQ ID NO: 3, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 3, whereby the endolysin is functional, wherein the function comprises the ability to lyse the cell wall of Gardnerella. The catalytic domain is preferably located N-terminally within the (recombinant) endolysin, thereby referred to as “N-terminal catalytic domain”, even more preferably the (recombinant) endolysin further comprises a cell-wall binding region and the N-terminal catalytic domain is located N-terminally from the C-terminal cell-wall binding region within the (recombinant) endolysin.

The “cell-wall binding region” as used herein refers to a functional polypeptide, wherein the function comprises the ability to bind to the cell wall of Gardnerella. The cell-wall binding region may comprise or consist of one, two, three or more cell-wall binding domains. Cell wall binding domains are polypeptides that interact with and/or bind to bacteria cell walls and/or specific substrates within bacteria cell walls. In particular, the cell call binding domain described herein can preferably specifically bind to the cell wall (e.g., to the peptidoglycan) of Gardnerella spp., such as Gardnerella vaginalis sensu stricto, Gardnerella leopoldii, Gardnerella piotii and/or Gardnerella swidsinskii, preferably all of them. The cell-wall binding region is preferably located C-terminally within the (recombinant) endolysin, thereby referred to as “C-terminal cell-wall binding region”, even more preferably the C-terminal cell-wall binding region is located C-terminally from the N-terminal catalytic domain within the (recombinant) endolysin.

The endolysin to be used in the therapeutic uses of the present invention preferably comprises a cell-wall binding region comprising or consisting of at least one cell-wall binding domain selected from the group consisting of polypeptides comprising or consisting of the amino acid sequence of any one of SEQ ID NOs: 11 to 28, and any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 11 to 28. As shown in WO 2020/225335, the most active cell-wall binding region is “B10” (comprising the cell-wall binding domains of SEQ ID NOs: 23 and 24), followed by “B11” (comprising the cell-wall binding domains of SEQ ID NOs: 25 and 26). Thus, in a preferred aspect of the present invention the cell-wall binding domain(s) of is/are selected from the group consisting of polypeptides comprising or consisting of the amino acid sequence of any one of SEQ ID NOs: 23, 24, 25 and 26, and any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 23, 24, 25 and 26, whereby the endolysin is functional, wherein the function comprises the ability to lyse the cell wall of Gardnerella. The cell-wall binding region is preferably located C-terminally within the (recombinant) endolysin, thereby referred to as “C-terminal cell-wall binding region”, even more preferably the (recombinant) endolysin further comprises a catalytic domain and the catalytic domain is located N-terminally from the C-terminal cell-wall binding region within the (recombinant) endolysin.

The endolysin to be used in the therapeutic uses of the present invention comprises preferably two cell-wall binding domains (within the cell-wall binding region). In one preferred aspect of the present invention the cell-wall binding domains of the endolysin of the invention each consists of a polypeptide comprising or consisting of the amino acid sequence of any one of SEQ ID NOS: 23, 24, 25 and 26, and any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 23, 24, 25 and 26, whereby the endolysin is functional, wherein the function comprises the ability to lyse the cell wall of Gardnerella. In one even more preferred aspect of the present invention, the endolysin comprises a first cell-wall binding domain and a second cell-wall binding domain, wherein said first cell-wall binding domain is selected from the group consisting of SEQ ID NOs: 23 and 25, and said second cell-wall binding domain is selected from the group consisting of SEQ ID NOs: 24 and 26. Preferably, said first cell-wall binding domain is located N-terminally of said second cell-wall binding domain.

In one more preferred embodiment, the endolysin to be used in the therapeutic uses of the present invention comprises
(i) a N-terminal catalytic domain consisting of a polypeptide which comprises or consists of the amino acid sequence of SEQ ID NO: 3, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 3; and
(ii) a C-terminal cell-wall binding region comprising or consisting of a first cell-wall binding domain and a second cell-wall binding domain,
wherein said first cell-wall binding domain is selected from the group consisting of SEQ ID NOs: 23 and 25, and any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 23 and 25, and
wherein said second cell-wall binding domain is selected from the group consisting of SEQ ID NOS: 24 and 26 and any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 24 and 26;
   whereby the endolysin is functional, wherein the function comprises the ability to lyse the cell wall of Gardnerella. Preferably, said first cell-wall binding domain is located N-terminally of said second cell-wall binding domain.

In one particularly preferred embodiment, the endolysin to be used in the therapeutic uses of the present invention comprises
(i) a N-terminal catalytic domain consisting of a polypeptide which comprises or consists of the amino acid sequence of SEQ ID NO: 3; and
(ii) a C-terminal cell-wall binding region comprising or consisting of a first cell-wall binding domain and a second cell-wall binding domain,
wherein said first cell-wall binding domain is selected from the group consisting of SEQ ID NOs: 23 and 25, and said second cell-wall binding domain is selected from the group consisting of SEQ ID NOs: 24 and 26.

Preferably, said first cell-wall binding domain is located N-terminally of said second cell-wall binding domain.

Illustrative examples of particularly preferred endolysins for use in the therapeutic uses of the present invention are “H2B10” (comprising from N-terminal to C-terminal: SEQ ID NOs: 3, 23, and 24), “H2B11” (comprising from N-terminal to C-terminal: SEQ ID NOs: 3, 25, and 26) as defined in WO 2020/225335, and “H2B10B11” (comprising from N-terminal to C-terminal: SEQ ID NOs: 3, 23, and 26).

The endolysin to be used in the therapeutic uses of the present invention further preferably comprises a linker region between the N-terminal catalytic domain and the C-terminal cell-wall binding region. The linker region may consist of a polypeptide having a length of 6 to 18 amino acids, preferably a length of 9 to 15 amino acids, even more preferably a length of 12 amino acids. Preferably, the linker region may consist of a polypeptide comprising or consisting of the amino acid sequence (i) (XXX)n, wherein each X can be independently G, A or S, preferably wherein the amino acid sequence (XXX)n is (GGS)n, wherein n corresponds to the number of repetitions of the sequence XXX, preferably wherein n is 2, 3, 4, 5 or 6, or (ii) X₁X₂GLNGX₃X₄NGGS (SEQ ID NO: 36), wherein X₁ is N or K, X₂ is A or V, X₃ is Y or C and X₄ is K or Q. Non-limiting examples of such linker regions are provided in SEQ ID NOs: 29 to 35.

In a particular preferred embodiment, the endolysin for use in the therapeutic uses of the present invention is “H2B10”, the sequence of which is set forth in SEQ ID NO: 1 or is “H2B10B11” the sequence of which is set forth in SEQ ID NO: 37. Thus, the invention provides a Gardnerella-specific endolysin, preferably a recombinant Gardnerella-specific endolysin, for use in treating a bacterial vaginosis, wherein the endolysin is to be administered to a patient who previously failed a treatment with antibiotics and/or who suffers from bacterial vaginosis wherein the infective bacteria are resistant to antibiotics treatment, wherein said endolysin is a polypeptide having at least 80% sequence identity with the amino acid sequence as provided in SEQ ID NO: 1 and having a killing activity against Gardnerella. In one more preferred embodiment, the invention provides a Gardnerella-specific endolysin, preferably a recombinant Gardnerella-specific endolysin, for use in treating a bacterial vaginosis, wherein the endolysin is to be administered to a patient who previously failed a treatment with antibiotics and/or who suffers from bacterial vaginosis wherein the infective bacteria are resistant to antibiotics treatment, wherein said endolysin is a polypeptide having at least 90% sequence identity with the amino acid sequence as provided in SEQ ID NO: 1 and having a killing activity against Gardnerella. In one even more preferred embodiment, the invention provides a Gardnerella-specific endolysin, preferably a recombinant Gardnerella-specific endolysin, for use in treating a bacterial vaginosis, wherein the endolysin is to be administered to a patient who previously failed a treatment with antibiotics and/or who suffers from bacterial vaginosis wherein the infective bacteria are resistant to antibiotics treatment, wherein said endolysin is a polypeptide having at least 95% sequence identity with the amino acid sequence as provided in SEQ ID NO: 1 and having a killing activity against Gardnerella. In one even more preferred embodiment, the invention provides a Gardnerella-specific endolysin, preferably a recombinant Gardnerella-specific endolysin, for use in treating a bacterial vaginosis, wherein the endolysin is to be administered to a patient who previously failed a treatment with antibiotics and/or who suffers from bacterial vaginosis wherein the infective bacteria are resistant to antibiotics treatment, wherein said endolysin is a polypeptide having at least 99% sequence identity with the amino acid sequence as provided in SEQ ID NO: 1 and having a killing activity against Gardnerella. In one most preferred embodiment, the invention provides a Gardnerella-specific endolysin, preferably a recombinant Gardnerella-specific endolysin, for use in treating a bacterial vaginosis, wherein the endolysin is to be administered to a patient who previously failed a treatment with antibiotics and/or who suffers from bacterial vaginosis wherein the infective bacteria are resistant to antibiotics treatment, wherein said endolysin comprises or consists of the amino acid sequence as provided in SEQ ID NO: 1. The endolysin comprising or consisting of the amino acid sequence as provided in SEQ ID NO: 37 may be also be employed in context of this invention, i.e. in context of the medical use of a (recombinant) Gardnerella-specific endolysin in treating a bacterial vaginosis, wherein the endolysin is to be administered to a patient who previously failed a treatment with antibiotics and/or who suffers from bacterial vaginosis wherein the infective bacteria are resistant to antibiotics treatment. Accordingly, the endolysin comprising or consisting of the amino acid sequence as provided in SEQ ID NO: 37 is an alternative to the Gardnerella-specific endolysin provided in SEQ ID NO. 1.

The terms “peptide”, “polypeptide”, “protein” and variations of these terms refer to peptide, oligopeptide, oligomer or protein including fusion protein, respectively, comprising at least two amino acids joined to each other by a normal or modified peptide bond, such as in the cases of the isosteric peptides, for example. These terms also include herewith “peptidomimetics” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. A peptide or polypeptide can be composed of amino acids other than the 20 amino acids defined by the genetic code. It can be composed of L-amino acids and/or D-amino acids. A peptide or polypeptide can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain or even at the carboxy- or amino-terminal ends. A peptide or polypeptide can be branched following an ubiquitination or be cyclic with or without branching. This type of modification can be the result of natural or synthetic post-translational processes that are well known to a person skilled in the art. For example, peptide or polypeptide modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature and well-known by the killed person of the art.

As defined herewith the terms “binding” and “bind to” referring to the binding capacity of an endolysin to the cell wall of a particular bacteria refers to the ability of said endolysin to specifically interact and adhere to the cell wall of said bacteria. The binding capacity of an endolysin to the cell wall of a bacteria can be determined by methods know of the art.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treating” as used herein covers any treatment of a bacterial vaginosis in a mammal, particularly a human, and includes: (a) preventing bacterial vaginosis from occurring in a patient which may be predisposed to bacterial vaginosis but has not yet been diagnosed as having it; (b) inhibiting bacterial vaginosis, i.e., arresting its development; or relieving the bacterial infection, i.e., causing regression of the bacterial infection and/or its symptoms or conditions such as improvement or remediation of damage. In particular, treatment of bacterial vaginosis comprises preventing, decreasing or even eradicating the infection, for instance by killing the infective bacteria and, thus, controlling, reducing or inhibiting bacterial proliferation as well as reducing the number of viable bacterial cells. Herein it is preferred that the disease, i.e. BV, is treated therapeutically in terms of a partial or complete cure of the disease or the symptoms.

The terms “patient” and “subjects” are used herein interchangeably and refer to mammals. For examples, mammals contemplated by the present invention include human, primates, domesticated animals such as cattle, sheep, pigs, horses, laboratory rodents and the like. It is preferred that the patient is a human being. It is even more preferred that the patient is a woman (also referred herein as a “female subject”).

The term “variant” refers to a polypeptide including insertions, deletions, and/or substitutions, either non-conservative or preferably conservative, relative to the native amino acid sequence. For example, the polypeptide may comprise an amino acid sequence with at least 80% identity to the native amino acid sequence, preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity to said amino acid sequence. Percent identity can be determined by methods well known in the art, using suitable computer programs for example MatGAT 2.0 (Myers and Miller, CABIOS (1989). Preferably, % identity is identified over the whole lengths of the sequences to be compared. It will be appreciated that percent identity is calculated in relation to polypeptides whose sequence has been aligned optimally. Fragment and variants of an amino acid sequence may be made using any of the methods of protein engineering, directed evolution and/or site-directed mutagenesis well known in the art (for example, see Molecular Cloning: a Laboratory Manual, 3rd edition, Sambrook & Russell, 2001, Cold Spring Harbor Laboratory Press). It will be appreciated by skilled persons that a polypeptide according to the invention, or fragment, variant, or fusion thereof, may comprise or consist of a derivative of a native amino acid sequence, or a fragment or variant thereof. Chemical derivatives of one or more amino acids may be achieved by reaction with a functional side group. Such derivatized molecules include, for example, those molecules in which free amino acid groups have been derivatized to form amine hydrochlorides, p-toluene sulphonyl groups, carboxybenzoxy groups, f-butyloxycarbonyl groups, chloroacetyl groups or formyl groups. Free carboxyl groups may be derivatized to form salts, methyl and ethyl esters or other types of esters and hydrazides. Free hydroxyl groups may be derivatized to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g., acetylation or thioglycolic acid amidation), terminal carboxylamidation (e.g., with ammonia or methylamine), and the like terminal modifications. It will be further appreciated by persons skilled in the art that peptidomimetic compounds may also be useful. Thus, by ‘polypeptide’ we include peptidomimetic compounds which exhibit endolysin activity. The term ‘peptidomimetic’ refers to a compound that mimics the conformation and desirable features of a particular polypeptide as a therapeutic agent.

Methods for the production of endolysins, or a fragment, variant, fusion, or derivative thereof, for use according to the invention are well known in the art. Conveniently, the endolysin for uses of the invention, or fragment, variant, fusion or derivative thereof, is or comprises a recombinant endolysin. The endolysin for use according to the invention can be produced by standard techniques of genetic engineering comprising the use of a recombinant vector comprising a polynucleotide encoding an endolysin as described herewith. Numerous expression systems can be used including bacterial plasmids and derived vectors, transposons, yeast episomes, insertion elements, yeast chromosome elements, viruses such as baculovirus, papilloma viruses such as SV40, vaccinia viruses, adenoviruses, fox pox viruses, pseudorabies viruses, retroviruses, cosmid or phagemid derivatives. The nucleotide sequence can be inserted in the recombinant expression vector by methods well known to a person skilled in the art such as, for example, those that are described in MOLECULAR CLONING: A LABORATORY MANUAL, Sambrook et al., 4th Ed., Cold Spring Harbor Laboratory Press, Cold Spring Harbor, N.Y., 2001. The recombinant vector can include nucleotide sequences that control the regulation, the expression, the transcription, and/or the translation of the polynucleotide encoding the endolysin, these sequences being selected according to the host cells that are used. The recombinant vector can further include nucleotide sequences such as those encoding His tags for facilitating the purification step. Subsequently, such a recombinant vector is introduced in a host cell according to methods that are well known to a person skilled in the art, such as those described in BASIC METHODS IN MOLECULAR BIOLOGY, Davis et al., 2nd ed., McGraw-Hill Professional Publishing, 1995, and MOLECULAR CLONING: A LABORATORY MANUAL, supra, such as transfection by calcium phosphate, transfection by DEAE dextran, transfection, microinjection, transfection by cationic lipids, electroporation, transduction or infection. The host cell can be, for example, bacterial cells such as E. coli, cells of fungi such as yeast cells and cells of Aspergillus, Streptomyces, insect cells, Chinese Hamster Ovary cells (CHO), C127 mouse cell line, BHK cell line of Syrian hamster cells, Human Embryonic Kidney 293 (HEK 293) cells. Preferably, the host cell is E. coli. Said host cells are then cultivated in appropriate conditions so as to produce the endolysin described herewith, which can then further be purified from the culture medium or from the host cell lysate by any standard purification methods including, Immobilized-Metal Affinity Chromatography (IMAC) (Block et al. 2008, Protein Expr. Purif. 27, 244-254).

The endolysin to be used in the therapeutic uses of the present invention preferably exhibit a low or non-observable resistance profile. Resistance of bacteria to anti-bacterial agents can be measured by assays described herein and known to those skilled in the art.

The endolysin to be used in the therapeutic uses of the present invention can be provided as compositions, for example a pharmaceutical composition comprising the endolysin described herein for uses of the present invention. Thus, also provided by the present invention is a pharmaceutical composition comprising the Gardnerella-specific endolysin, preferably the recombinant Gardnerella-specific endolysin, as described herein and optionally a pharmaceutically acceptable carrier and/or diluent for uses in treating a bacterial vaginosis as described herein.

The term “pharmaceutical composition” refers to a preparation which is in such a form as to permit biological activity of the active ingredient(s) to be unequivocally effective and which contains no additional component which would be toxic to patients to which the said composition would be administered. As used herein, “pharmaceutical composition” means a therapeutically effective formulation for use in the methods of the invention. A “therapeutically effective amount”, or “effective amount”, or “therapeutically effective”, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce, and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art. In one embodiment of the uses of the invention, the pharmaceutical composition comprises a recombinant Gardnerella-specific endolysin as described herein above and is for use in treating bacterial vaginosis as described herein above. Thus, the pharmaceutical composition may comprise an amount of an endolysin, or fragment, variant, fusion or derivative thereof, sufficient to inhibit at least in part the growth of cells of the genus Gardnerella in a patient as described herein above who is infected or susceptible to infection with such cells. Preferably, the pharmaceutical composition comprises an amount of endolysin, or fragment, variant, fusion or derivative thereof, sufficient to kill cells of the genus Gardnerella in the patient to be treated as defined herein above. It will be appreciated by persons skilled in the art that the endolysins to be used according to the invention are generally administered in admixture with a suitable pharmaceutical excipient, diluent or carrier selected with regard to the intended route of administration and standard pharmaceutical practice (for example, see Remington: The Science and Practice of Pharmacy, 19th edition, 1995, Ed. Alfonso Gennaro, Mack Publishing Company, Pennsylvania, USA). For example, the endolysins can be administered locally, i.e. locally into the vagina of a female subject and/or, in a male subject into or on the glans penis, prepuce or urethral entry. Herein the term “(administration) into or on the glans penis” also includes “(administration) into and on the glans penis”. In line with this, the term “(administration) into or on the glans penis, prepuce or urethral entry of a male subject” also includes “(administration) into and on the glans penis and on the prepuce and on the urethral entry of a male subject”. In another embodiment, the endolysins can be co-administered with a compound or composition which adjusts the pH of the vagina. In some embodiment the compound or composition adjusts the pH of the vagina to pH 4.0 to 6.0, preferably to pH 5.0.

The term “pharmaceutically acceptable” refers to a carrier comprised of a material that is not biologically or otherwise undesirable.

The term “carrier” refers to any components present in a pharmaceutical formulation other than the active agent and thus includes diluents, binders, lubricants, disintegrants, fillers, coloring agents, wetting or emulsifying agents, pH buffering agents, preservatives and the like.

Compositions for uses of the invention can contain one or more endolysin polypeptides. In this embodiment, endolysin polypeptides can either be present as independent polypeptides or as fusion proteins comprising said endolysin polypeptides or fragments thereof.

Pharmaceutical compositions for uses of the invention may further comprise one or more pharmaceutically acceptable additional ingredient(s) such as alum, stabilizers, antimicrobial agents, buffers, coloring agents, flavoring agents, adjuvants, and the like. It is preferred that the pharmaceutical composition for uses of the invention does not comprise imidazole.

The Gardnerella-specific endolysin, preferably the recombinant Gardnerella-specific endolysin for the medical uses and for the methods of treatment as provided herein, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of a suppository for local (including intra-vaginal) use. Yet, also other means of administration are envisaged in context of this invention. Such means may comprise, inter alia, parenteral administration(s). Also pharmaceutical compositions for topical and/or local administration are envisaged, for example pharmaceutical compositions in form of (topical) gels, lotions or creams or via a pessary, i.e. a vaginal suppository. Such a pessary/vaginal suppository may be coated with the Gardnerella-specific endolysin, preferably the recombinant Gardnerella-specific endolysin, as described herein. The pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed. Compositions for uses of the invention may also be liquid formulations including, but not limited to, aqueous or oily suspensions, solutions, emulsions, syrups, and elixirs. The compositions may also be formulated as a dry product for reconstitution with water or other suitable vehicle before use. Such liquid preparations may contain additives including, but not limited to, suspending agents, emulsifying agents, non-aqueous vehicles and preservatives. Suspending agents include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid. Further materials as well as processing techniques and the like are set out in Part 5 of Part 5 of Remington's “The Science and Practice of Pharmacy”, 22nd Edition, 2012, University of the Sciences in Philadelphia, Lippincott Williams & Wilkins.

Solid compositions for uses of the invention may be in the form of tablets or lozenges formulated in a conventional manner. Tablets may be coated according to methods well known in the art. Injectable compositions are typically based upon injectable sterile saline or phosphate-buffered saline or other injectable carriers known in the art.

Compositions for uses of the invention may also be formulated as suppositories, which may contain suppository bases including, but not limited to, cocoa butter or glycerides. Compositions of this invention may also be formulated transdermal formulations comprising aqueous or non-aqueous vehicles including, but not limited to, creams, ointments, lotions, pastes, medicated plaster, patch, or membrane. Compositions for uses of the invention may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

Compositions for uses of the invention may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

The compounds for uses of the invention can also be administered in sustained release forms or from sustained release drug delivery systems. A description of representative sustained release materials can also be found in Remington's “The Science and Practice of Pharmacy”.

Also provided by the present invention is a method of treating a bacterial vaginosis as described herein above in a patient who previously failed a treatment with antibiotics and/or who suffers from bacterial vaginosis wherein the infective bacteria are resistant to antibiotics treatment, wherein the method comprises administering to said patient a therapeutically effective amount of a Gardnerella-specific endolysin, preferably a recombinant Gardnerella-specific endolysin, as described herein or a pharmaceutical composition comprising a Gardnerella-specific endolysin, preferably recombinant Gardnerella-specific endolysin, as described herein.

Embodiments and definitions described herein above for the therapeutic uses according to the invention apply in the context of the method of treating BV mutatis mutandis.

In one embodiment of the uses of the invention, a host cell or pharmacological composition comprising a host cell is used to deliver the endolysin (preferably a host cell).

It will be appreciated that the endolysins and pharmaceutical compositions for uses as described herein may be administered to a subject in combination with one or more additional therapeutic agents. For example, the endolysins and pharmaceutical compositions described herein may be administered to a subject in combination with:
(a) one or more conventional antibiotic treatments. Such antibiotics may include Clindamycin, Metronidazole or any other suitable antibiotics known by a skilled person in the art;
(b) one or more additional endolysins, or nucleic acid molecules, vectors, host cell or bacteriophage capable of expressing the same;
(c) a compound or composition which adjusts the pH of the vagina, preferably to pH 4.0 to 6.0, more preferably to about pH 5.0. Such pH adjusting compounds may include phosphate, lactic acid (e.g. the natural acidification substance which Lactobacilli secrete to establish an acidic milieu) or other organic acids, e.g. carboxy-substituted polymers;
(d) a therapy to neutralize the toxins released upon bacterial lysis of G.vaginalis cells within the vagina. Suitable neutralizing therapies may include antibodies (see Babcock et al., 2006, Infect. Immun. 74:6339-6347) and toxin absorbing agents such as tolevamer (see Barker et al., 2006, Aliment. Pharmacol. Ther. 24:1525-1534)
(e) a probiotic.

The present invention is further described by reference to the following non-limiting figures and examples.

The Figures show:

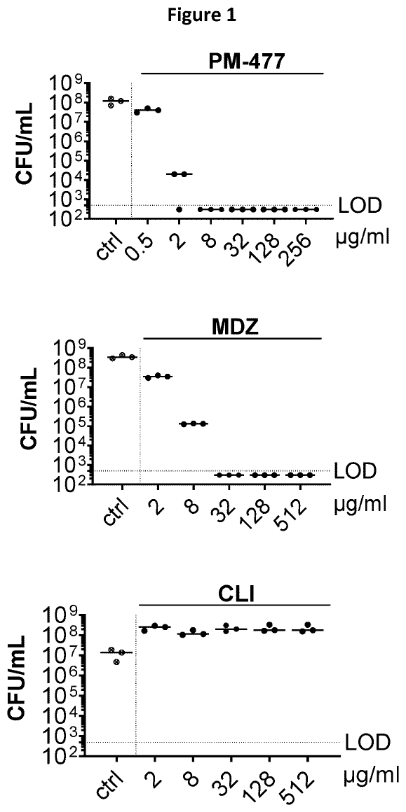

FIG. 1. Reduction in biofilm CFU of a representative Gardnerella strain (G. vaginalis ATCC14018) by MDZ, CLI, and PM-477 (H2B10)

FIG. 1 shows that G. vaginalis (ATCC14018) grown in biofilm can be eradicated by MDZ and PM-477 (H2B10), but not by CLI treatment. Biofilms were grown for 40 h and then incubated for another 24 h with the indicated antimicrobial. For analysis, the antimicrobials were washed off, the biofilm was dislodged mechanically, and the surviving cells enumerated (in CFU/mL) by quantitative plating. LOD, limit of detection. Abbreviations: ctrl: control, medium only; LOD: limit of detection.

FIG. 2. Resistance formation by serial passaging

FIG. 2 shows that six Gardnerella strains were passaged for up to 25 days in the presence of MDZ or MDZ-OH at sub-MIC concentrations, as indicated in the legend. One representative Gardnerella strain was passaged with PM-477 (H2B10) for 25 days as indicated in the figure legend. MIC (μg/mL) was determined every day prior to the next round of passaging

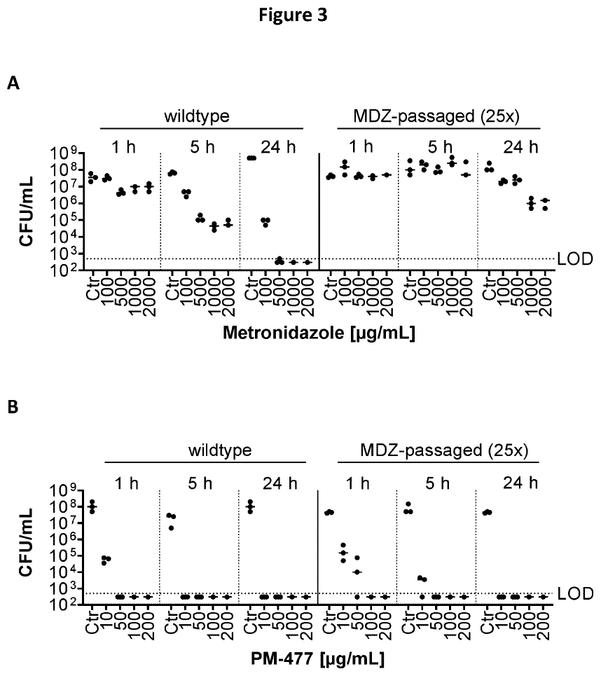

FIG. 3. Lytic effect of MDZ and PM-477 (H2B10) on wildtype and MDZ-passaged G. vaginalis (ATCC 14018^{T}) cells in suspension

FIG. 3 shows wild type cells naïve to MDZ and the strain passaged with sub-MIC concentrations of MDZ for 25 rounds were treated with for 1, 5 and 24 h with MDZ (A) and PM-477 (H2B10) (B), respectively. Abbreviations: ctr: control, medium only; LOD: limit of detection.

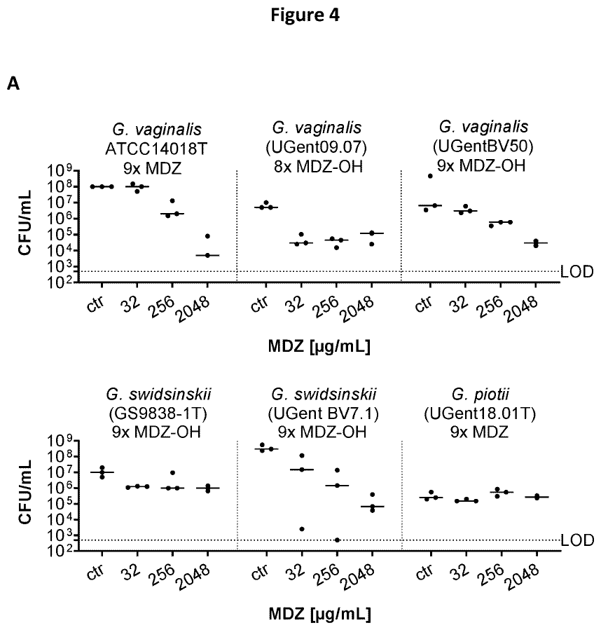

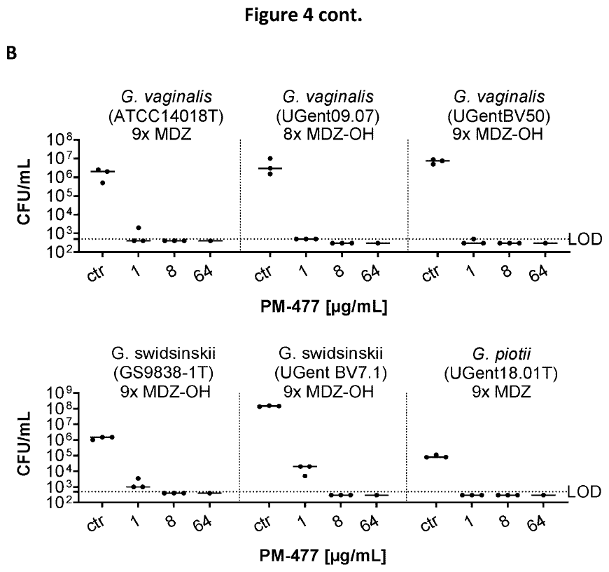

FIG. 4. MDZ-passaged strains grown as biofilm are resistant to MDZ and susceptible to PM-477 (H2B10)

FIG. 4 shows that the 72 h pre-formed biofilms were treated for 24 h with different concentrations of MDZ (A) and PM-477 (H2B10) (B), as indicated on the x-axis, and surviving cells were quantified. Abbreviations: ctr: control, medium only; LOD: limit of detection; MDZ-OH: hydroxy metronidazole. The numbers indicate the rounds of passaging with sub-MIC concentrations of MDZ or MDZ-OH.

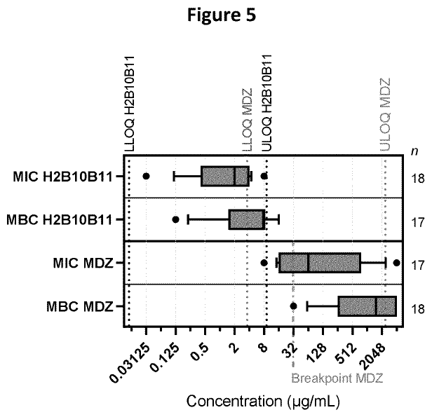

FIG. 5: Lytic effect of H2B10B11 is maintained on MDZ-resistant Gardnerella isolates.

FIG. 5 shows MIC and MBC99.5 values of H2B10B11 and MDZ determined on a panel of n=18 Gardnerella BV patient isolates. MBC_{99.5} of H2B10B11 could not be analyzed for one strain and another strain's growth was too low to determine the MIC of MDZ (n=17). For both antimicrobials the same strains were tested. Box (25-75 percentile) and whiskers (10-90 percentile) are shown, the median is indicated as line, data points below and above the whiskers are indicated as circles. The breakpoint of resistance of MDZ is indicated as line (32 μg/mL). Values above ULOQ are arbitrarily displayed as 16 μg/mL (H2B10B11) and as 4096 μg/mL (MDZ). Abbreviations: LLOQ: lower limit of quantification; ULOQ: upper limit of quantification.

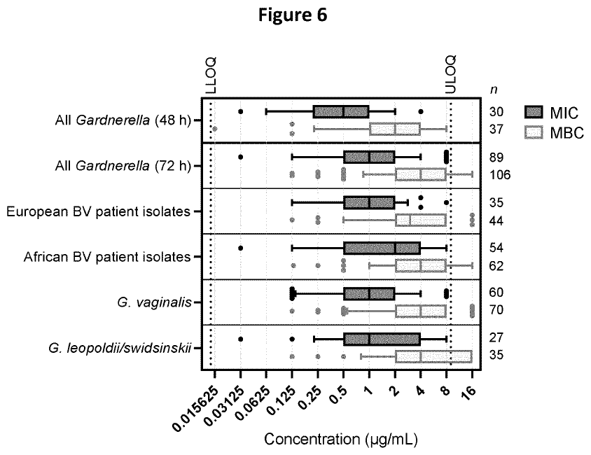

FIG. 6: Gardnerella BV patient isolates of various origins are susceptible to H2B10B11.

FIG. 6 shows MIC and MBC_{99.5} values of H2B10B11 determined on Gardnerella BV patient isolates received as stated in Table 1 or isolated in house. Number n of analyzed strains is indicated at the right site of the graph. The MIC for a subset of fast-growing strains was determined after 48 h. The MIC of most of the strains was determined after 72 h, because the majority of the clinical isolates are poorly growing at pH 5.5. Box (25-75 percentile) and whiskers (10-90 percentile) are shown, the median is indicated as line, data points below and above the whiskers are indicated as circles. Values above ULOQ are displayed as 16 μg/mL. Abbreviations: LLOQ: lower limit of quantification; ULOQ: upper limit of quantification.

The following Examples illustrate the invention.

### Overview

Antibiotics are the mainstay of therapy for bacterial vaginosis (BV). However, the rate of treatment failure in patients with recurrent BV is about 50%. Herein, the present inventors investigated potential mechanisms of therapy failure, including the propensity of resistance formation and biofilm activity of metronidazole (MDZ), clindamycin (CLI) and PM-477 (H2B10), a genetically engineered endolysin with specificity for bacteria of the genus Gardnerella. Determination of the minimum inhibitory concentration (MIC) indicated that 60% of a panel of 22 Gardnerella isolates of four different species were resistant to MDZ, while all strains were highly susceptible to CLI and to the endolysin PM-477 (H2B10). Similar results were obtained with a panel of 18 Gardnerella isolates from vaginal swabs of BV patients, 10 of which were resistant to MDZ (MIC>32 μg/mL) but inhibited by 4-8 μg/mL of the endolysin H2B10B11 (MIC₉₀=4.4 μg/mL).

Six strains, all of which were initially susceptible to MDZ, were passaged with MDZ or its more potent hydroxy metabolite. All of them generated full resistance after 5-10 passages, resulting in MICs>512 μg/mL. In contrast, only a mild increase in MIC was found for PM-477 (H2B10). There was also no cross-resistance formation, as MDZ-resistant Gardnerella strains remained highly susceptible to PM-477 (H2B10) both in suspension and in pre-formed biofilms. Strains that were resistant to MDZ in suspension were also tolerant to MDZ at >2048 μg/mL when growing as biofilm. All strains were susceptible to PM-477 (H2B10) when grown as pre-formed biofilms, at minimum biofilm eradicating concentrations (MBEC) in the range of 1-4 μg/mL. Surprisingly, the MBEC of CLI was >512 μg/mL for 7 out of 9 tested Gardnerella strains, all of which were susceptible to CLI when growing in suspension. The observed challenges of MDZ and CLI due to resistance formation and ineffectiveness on biofilm, respectively, could be one explanation for the frequent treatment failures in uncomplicated or recurrent BV. Therefore, the high efficacy of PM-477 (H2B10) in eliminating Gardnerella in in vitro biofilms as well as its high resilience to resistance formation, makes PM-477 (H2B10) a promising alternative for the treatment of bacterial vaginosis, especially in patients with frequent recurrence. Moreover, the inventors examined the MIC of H2B10B11 on a total of 104 Gardnerella isolates from different geographical origins as well as several type strains and did not find a significant difference in their susceptibility towards H2B10B11.

### Materials and Methods

### Bacterial Isolates and Culture Conditions

Gardnerella spp. isolates of different species (i.e., G. vaginalis sensu stricto, G. leopoldii, G. piotii, and G. swidsinskit, cf. Vaneechoutte et al., 2019, Int. J. Syst. Evol. Microbiol. 69, 679-687) were obtained from the Laboratory of Bacteriology, University of Ghent, Belgium. These isolates include strains purchased from culture collections and fresh isolates from BV patients obtained from the University Clinic Bruges. Gardnerella isolates were grown on chocolate (Choc) agar plates (Becton Dickinson) under anaerobic conditions in an anaerobic chamber equipped with anaerobic atmosphere generation bags (Sigma Aldrich) for 48 h. All isolates were cultured in New York City broth III (NYCB), consisting of 10 mM HEPES (Sigma Aldrich), 15 g/L Proteose Peptone (Sigma Aldrich), 3.8 g/L yeast extract (Thermo Fisher Scientific), 86 mM sodium chloride (Carl Roth), 28 mM a-D-glucose (Sigma Aldrich), supplemented with 10% horse serum (HS) (Thermo Fisher Scientific). Table 1 lists all Gardnerella isolates studied. For enabling disclosure purposes, at least one strain of each identified species of the genus Gardnerella, namely G. vaginalis sensu stricto, G. leopoldii, G. piotii, and G. swidsinskii according to the new nomenclature following Vaneechoutte et al., 2019, Int. J. Syst. Evol. Microbiol. 69, 679-687, is publicly available to the skilled person.

These publicly available strains are identified in Table 1 below with a “T” as type strain (T) for the corresponding Gardnerella species. As illustrative examples, ATCC 14018 is a publicly available type strain of the G. vaginalis sensu stricto, UGent 18.01 is a publicly available type strain of G. piotii, UGent 06.41 and UGent 09.48 are both publicly available type strains of G. leopoldii, and GS 9838-1 is a publicly available type strain of G. swidsinskii. As is evident to a person skilled in the art, also other bacterial strains from each species may be used. Moreover, further Gardnerella strains were obtained via isolation from vaginal swabs from BV patients received from several medical centres, namely from Ghent (BE), Vilnius (LT), Vienna (AT) and Durban (ZA). All samples were obtained under informed consent and with ethical approval by AZ St. Jan (Bruges, BE), the Lithuanian Bioethics Committee (LT), the Medical University in Vienna (AT) and University of KwaZulu-Natal (Durban, ZA). The swabs were streaked onto Choc or Gardnerella-selective agar plates (both Becton Dickinson). Single strains were isolated by picking individual colonies, re-streaking on Choc or Gardnerella-selective agar plates (both Becton Dickinson), incubating the plates anaerobically for 48 h at 37° C., and repeating the isolation and re-streak process for at least 2 further times. After this purification of individual strains,, glycerol cryo stocks were prepared and the strains were submitted for identification via MALDI-TOF MS (Bruker Biotyper). With this method, G. vaginalis could be distinguished from G. leopoldii and G. swidsinskii, however, there is no discrimination between the latter two possible. Also, G. piotii could not be identified to the species level.

### Preparation of PM-477 (Also Referred to as “H2B10” Interchangeably)

The endolysin PM-477 (H2B10, SEQ ID NO:1) was engineered and produced as previously described (Landlinger et al., (2021 Pathogens 10, 1-19) and WO 2020/225335 A1). The endolysin H2B10B11 differs from H2B10 in one amino acid exchange in one of the binding domains (see SEQ ID NO: 37). Briefly, PM-477 (H2B10) and H2B10B11 were recombinantly expressed in Escherichia coli BL21 (DE3). Protein purification was performed by affinity chromatography on a nickel-nitrilotriacetic acid (Ni-NTA) HISTrap column. The protein was eluted with 50 mM MES (Carl Roth) pH 7, 150 mM NaCl (Carl Roth), 150 mM-500 mM imidazole fractions (two-fold dilutions). Compared to the preparation used in the study of Landlinger et al. (2021, Pathogens 10, 1-19), the N-terminal His-tag was cleaved off by digestion with 1:100 w/w 3C protease. The removed tag and the protease were separated from PM-477 (H2B10) and H2B10B11 by anion exchange chromatography. The untagged proteins were concentrated (if needed) and dialyzed against MES buffer (50 mM MES pH 5.5, 200 mM NaCl, 8 mM MgSO₄ (Sigma Aldrich)).

The protein concentration was determined at OD 260/280 nm or by using the Pierce™ BCA (bicinchoninic acid) protein assay kit (Thermo Fisher Scientific). Purified aliquots of 1000 uL comprising 0.7 mg/mL PM-477 (H2B10) or 1.3 mg/mL H2B10B11 were stored at −80° C. till the moment of use.

The amino acid sequence of PM-477 (H2B10) is set forth in SEQ IQ NO: 1. The amino acid sequence of H2B10B11 is set forth in SEQ IQ NO: 37.

### Culture-Based Assessment of Bactericidal Activity

Bacterial suspensions (OD₆₀₀ of 0.1, corresponding to approximately 10⁷-10⁸ CFU/mL) were prepared by scraping the cells from confluently grown agar plates and diluting them into NYCB+10% HS, pH 5.5. Reactions were performed in triplicate by mixing ten μl of endolysin (200 μg/mL) with 90 μL bacterial suspension in the wells of a 96-well plate. Ten UL MES buffer without the endolysin was used as a control. The 96-well reaction plate was incubated anaerobically at 37° C. for 5 h. Tenfold dilution series (10⁻¹ to 10⁻⁶) of the cell reaction mixtures were prepared in NYCB+10% HS and 2 μL of each dilution were spotted onto Choc agar plates. After anaerobic incubation at 37° C. for 48 h, colonies were counted, CFU/mL calculated, and the log₁₀ reduction compared to MES buffer treated control was determined.

### MIC Assessment

The minimum inhibitory concentration (MIC), which is a standard measure of the activity of antimicrobials, was determined according to the Clinical and Laboratory Standards Institute protocol (2018) Methods for Antimicrobial Susceptibility Testing of Anaerobic Bacteria (Carpenter et al., 2018). Bacterial suspensions of 10⁵ to 10⁶ CFU/mL in NYCB+10% HS were treated with a two-fold dilution series of either PM-477 (H2B10) or of the antibiotic clindamycin (Clindamycin hydrochloride, Sigma Aldrich), both tested at a starting concentration of 64 μg/mL, or the antibiotics metronidazole (MDZ; Gatt-Koller) and its hydroxy metabolite 1(2-hydroxyethyl)-2-hydroxy-methyl-5-nitroimidazole (MDZ-OH; Sigma Aldrich), starting at concentrations of 2048 or 512 ug/mL, respectively, or tinidazole starting at 128 μg/mL (TDZ, Sigma Aldrich). Controls for growth in the absence of antimicrobials were also included. OD₆₀₀ was recorded by a microplate reader (Tecan, Grodig, Austria) after incubation at 37° C. for 48 h or, for some fast-growing strains, after incubation for 24 h. Absence of growth was defined as OD₆₀₀≤0.14. Since most of the clinical isolates summarized in FIG. 5 and FIG. 6 grew slowly and poorly at pH 5.5, overnight cultures in NYCB (unadjusted pH around 7, instead of adjustment to pH 5)+10% HS were inoculated and used as input for MIC determination. MIC readout and MBC_{99.5} spotting were performed after 72 h incubation for H2B10B11. MBC_{99.5} is the minimum concentration of an antibacterial agent that results in bacterial death of all cells in suspension up to a defined limit of detection, resulting in killing of at least 99.5% of bacteria. To determine MBC_{99.5}, 2 μL of the antimicrobial dilution series and bacteria suspension in the MIC plate were spotted on NYCB+10% HS agar plates and incubated anaerobically for 2-3 days To bridge the MIC results determined after 72 h incubation with earlier obtained results, the MICs for a subset of fast-growing strains thereof were determined after 48 h incubation (FIG. 6).

### Serial Passaging for Resistance Formation Profiling

Bacterial suspensions of 10⁵ to 10⁶ CFU/mL in NYCB+10% HS were prepared for the treatment with a two-fold dilution series of either the endolysin PM-477 (H2B10) produced as described previously (Landlinger et al., 2021, Pathogens 10, 1-19), PM-477 (H2B10) with modifications defined herein, metronidazole (MDZ; Gatt-Koller), or 1(2-hydroxyethyl)-2-hydroxy-methyl-5-nitroimidazole (MDZ-OH; Sigma Aldrich). The final volume of the reaction was 100 UL and the reactions were performed in triplicate in 384-well plates. The OD₆₀₀ was measured using a microplate reader (Tecan, Grodig, Austria) to generate timepoint 0 h baseline values. The reactions were incubated anaerobically for 24 h, then the OD₆₀₀ was measured again, and the MIC was measured as the lowest concentration with OD₆₀₀<0.14. The bacterial cell suspension treated with the highest concentration of antimicrobial for which growth was detected (‘sub-MIC’) was diluted to 10⁵ to 10⁶ CFU/mL and treated anew with a dilution series of antimicrobials. This process was repeated for a maximum of 25 rounds. In each round, the input CFU/mL was determined by quantitative plating.

### Biofilm Formation and MBEC Determination

Gardnerella spp. (see figures for strain specification) was resuspended in brain heart infusion broth supplemented with 2% (w/v) gelatin, 0.5% yeast extract (w/v), 0.1% starch (w/v) and 0.25% glucose (w/v) (sBHIG). Passaged strains were resuspended in unbuffered NYCB supplemented with 1% glucose (w/v) (sNYCB). The OD₆₀₀ was set to 0.1 (approx. 10⁷-10⁸ CFU/mL) and the cell suspension was diluted 1:10 in growth medium as input for biofilm formation. A total of 200 μL of the respective bacterial suspensions was added to 96-well flat bottom plates (tissue-culture treated, Sigma-Aldrich). The biofilms were grown under anaerobic conditions at 37° C. for 40 to 72 hours, depending on the isolate. Subsequently, the supernatant was removed, and the biofilm was treated with antimicrobials dissolved in sBHIG (at pH 5 for treatment with PM-477 (H2B10), and unbuffered for treatment with antibiotics) in 100 μL and incubated anaerobically at 37° C. for another 24 hours. After the treatment, the biofilms were washed twice with 200 μL 1x PBS and dissolved by vigorous pipetting (40x up and down). Serial dilutions of the dissolved cells were spotted on chocolate agar plates. The plates were incubated anaerobically for two to three days and the minimum biofilm eradication concentration (MBEC) of the endolysin or the antibiotics in use were calculated.

### Statistical Analysis

Where appropriate, data were log-normalized prior to applying statistical tests (e.g., for CFU/mL values, and as indicated in the figure legends). When only two groups were compared, the unpaired two-tailed t-test was used as indicated in the respective figure legends. Multiple groups were compared by two-tailed one-way ANOVA tests. The software used for statistical analyses was GraphPad Prism8. Differences between groups were considered statistically significant when p<0.05.

### Example 1: 59% of Tested Gardnerella Type Strains and Patient Isolates are MDZ Resistant but Highly Susceptible to PM-477 (H2B10) and H2B10B11

The minimum inhibitory concentrations (MIC) of MDZ, tinidazole (TDZ), CLI, and PM-477 (H2B10) were determined for 22 Gardnerella strains following the Clinical and Laboratory Standards Institute (CLSI) protocol for anaerobic bacteria (Carpenter et al., 2018). The resistance breakpoints defined by EUCAST for Gram-positive anaerobes are >4 μg/mL for both CLI and MDZ (EUCAST, 2021). However, topical intravaginal antibiotic administration is recommended for BV therapy, which allows to reach concentrations of active ingredients in the mg/mL range. Therefore, in this example, resistance breakpoints of ≥8 μg/mL and ≥32 μg/mL for CLI and MDZ, respectively, was used as previously described for BV-related bacteria (Petrina et al., 2017, Anaerobe 47, 115-119). Susceptibility of Gardnerella strains to the antimicrobials PM-477 (H2B10), MDZ, CLI and TDZ was evaluated. MIC and MBC_{99.5} (minimum concentration that reduces CFU by 99.5% within 24 h of treatment) values are reported for each strain in Table 1 below. Abbreviations: n.d., not determined; SJHB, St. Jan Hospital Bruges.

TABLE 1

| | | | | Antimicrobials [μg/ml] | | | | | | | | | | | |
|---|---|---|---|---|---|---|---|---|---|---|---|---|---|---|---|
| Gardnerella strain/isolate | | | | PM-477 | | MDZ | | | | CLI | | TDZ | | | |
| Name | Origin | Strain | Clade | MIC | MBC_{99.5} | MIC | | MBC_{99.5} | | MIC | MBC_{99.5} | MIC | | MBC_{99.5} | |
| G. vaginalis (Gv9) | ATCC | ATCC 14018(T) | IB | 0.06 | 0.25 | 8 | | 16 | | 0.25 | 0.5 | 128 | (R) | >128 | (R) |
| G. vaginalis (Gv1) | UG | UGent 09.07 | IA | 0.25 | 1 | 64 | (R) | 512 | (R) | 0.25 | 0.25 | >128 | (R) | >128 | (R) |
| G. vaginalis (Gv5) | UG | UGent 09.01 | IB | <0.03 | 0.125 | 8 | | 512* | (R) | 0.13 | 0.25 | 4 | | 8 | |
| G. vaginalis (Gv8) | UG | UGent 25.49 | IB | 0.03 | 0.125 | 8 | | 32 | (R) | <0.06 | 0.25 | 4 | | 32 | (R) |
| G. vaginalis (BV50) | SJHB | UGent BV50.1 | n.d. | 0.5 | 1 | 32 | (R) | 128 | (R) | 0.25 | 0.5 | 64 | (R) | >128 | (R) |
| G. vaginalis (BV111) | SJHB | UGent BV111.5 | n.d. | 0.25 | 0.5 | 8 | | 64 | (R) | 0.125 | 2 | 4 | | 128 | (R) |
| G. vaginalis (FB049) | UG | UGent FB049-01 | n.d. | 0.25 | 0.25 | 16 | | 64 | (R) | 0.5 | 1 | 8 | | 64 | (R) |
| G. vaginalis (FB061) | UG | UGent FB061-03 | n.d. | 0.5 | 2 | 8 | | 32 | (R) | 0.25 | 1 | 4 | | 32 | (R) |
| G. leopoldii (Gl11) | UG | UGent 09.48 | II | 0.125 | 0.5 | 128 | (R) | >128 | (R) | 0.5 | 1 | 128 | (R) | >128 | (R) |
| G. leopoldii (BV217) | SJHB | UGent BV217.1 | n.d. | 0.25 | 1 | 256 | (R) | 512 | (R) | 0.5 | 1 | n.d. | | n.d. | |
| G. leopoldii (BV13) | SJHB | UGent BV13.2 | n.d. | 0.5 | 2 | >128 | (R) | >128 | (R) | 0.5 | 1 | >128 | (R) | 128 | (R) |
| G. piotii (Gp17) | UG | UGent 18.01(T) | III | 1 | 4 | 32 | (R) | 64 | (R) | 0.5 | 1 | 64 | (R) | >128 | (R) |
| G. piotii (Gp22) | UG | UGent 21.28 | III | 1 | 2 | 64 | (R) | >128 | (R) | 0.25 | 0.5 | >128 | (R) | >128 | (R) |
| G. piotii (P80275) | SJHB | UGent P80275 | III | 0.5 | 1 | 16 | | 64 | (R) | 0.5 | 1 | 32 | (R) | 128 | (R) |
| G. piotii (FB041) | SJHB | UGent FB041 | III | 0.5 | 1 | 32 | (R) | 64 | (R) | 1 | 2 | 64 | (R) | 128 | (R) |
| G. piotii (BV049) | SJHB | UGent BV049.1 | n.d. | 1 | 1 | 8 | | 32 | (R) | 0.125 | 0.125 | n.d. | | n.d. | |
| G. piotii (BV140) | SJHB | UGent BV140.2 | n.d. | 1 | 2 | 128 | (R) | >512 | (R) | 2 | 64 (R) | n.d. | | n.d. | |
| G. piotii (BV154) | SJHB | UGent BV154.1 | n.d. | 0.5 | 1 | 8 | | 32 | (R) | 0.5 | 0.5 | n.d. | | n.d. | |
| G. swidsinskii (Gs23) | UG | GS 10234 | IV | 0.063 | 0.125 | 64 | (R) | >128 | (R) | 0.25 | 0.25 | >128 | (R) | >128 | (R) |
| G. swidsinskii (Gs24) | UG | GS 9838-1(T) | IV | 0.03 | 0.06 | 256 | (R) | >512 | (R) | <0.06 | 0.5 | >128 | (R) | >128 | (R) |
| G. swidsinskii | SJHB | UGent BV139.3 | n.d. | 0.125 | 0.125 | 256 | (R) | 256 | (R) | 0.125 | 0.25 | n.d. | | n.d. | |
| (BV139) | | | | | | | | | | | | | | | |
| G. swidsinskii (BV7) | SJHB | UGent BV7.1 | n.d. | 0.25 | 0.25 | 256 | (R) | >512 | (R) | 0.5 | 1 | 128 | (R) | >128 | (R) |

(T) = T Strain

The minimum inhibitory concentrations (MIC) of MDZ, CLI and PM-477 (H2B10) for Gardnerella type strains and patient isolates are summarized in Table 2 below. Resistance is defined as ≥32 μg/mL and ≥8 μg/mL for MDZ and CLI, respectively. MIC₉₀ is defined as the MIC value for 90% of the tested strains. For PM-477 (H2B10), the resistance breakpoint is not defined, therefore a “% resistant” is not defined (n.d.).

TABLE 2

| | | | MIC (μg/mL] | | |
|---|---|---|---|---|---|
| | | | | | N resistant |
| Species | N | Antimicrobial Agent | Range | MIC₉₀ | (%) |
| Gardnerella vaginalis | 8 | MDZ | 8-64 | 64 | 2 (25%) |
| | | CLI | <0.06-0.5 | 0.5 | 0 |
| | | PM-477 (H2B10) | <0.03-0.5 | 0.5 | n.d. |
| Gardnerella leopoldii | 3 | MDZ | 128->256 | >256 | 3 (100%) |
| | | CLI | 0.5 | 0.5 | 0 |
| | | PM-477 (H2B10) | 0.125-0.5 | 0.5 | n.d. |
| Gardnerella piotii | 7 | MDZ | 8-128 | 128 | 4 (57%) |
| | | CLI | 0.125-2 | 2 | 0 |
| | | PM-477 (H2B10) | 0.5-1 | 1 | n.d. |
| Gardnerella swidsinskii | 4 | MDZ | 64->256 | >256 | 4 (100%) |
| | | CLI | <0.06-0.5 | 0.5 | 0 |
| | | PM-477 (H2B10) | 0.03-0.25 | 0.25 | n.d. |
| All Gardnerella | 22 | MDZ | 8->256 | >256 | 13 (59%) |
| | | CLI | <0.06-2 | 0.5 | 0 |
| | | PM-477 (H2B10) | <0.03-1 | 1 | n.d. |

For MDZ, MICs in the range of 8 to >256 μg/mL were observed, with a MIC value for 90% of strains (MIC₉₀) above 256 μg/mL. Thirteen out of 22 tested isolates (59%) had a MIC for MDZ at or higher than 32 μg/mL. With regards to the different Gardnerella species, MDZ resistance rates varied from 25% for G. vaginalis to 100% for G. leopoldii and G. swidsinskii. The MIC values for MDZ and TDZ were very similar, and largely the same isolates were resistant or susceptible, indicating very similar mechanisms of activity and resistance formation. Hence, because it is found that the resistance status across strains is comparable for MDZ and TDZ, without being bound by any theory, it is expected that these findings hold for all nitroimidazoles, including secnidazole. In contrast, all Gardnerella strains of all four species were highly susceptible to CLI (MIC₉₀=0.5 μg/mL, range <0.06-2 μg/mL) as well as to the endolysin PM-477 (H2B10) (MIC₉₀=1 μg/mL, range <0.03-1 μg/mL).

MIC and MBC_{99.5} of H2B10B11 and MDZ were determined on 18 in-house isolated Gardnerella strains from vaginal swabs of BV patients (FIG. 5). The majority of the analyzed isolates were resistant against MDZ (breakpoint of resistance 32 μg/mL) with a median MIC of 64 μg/mL and median MBC_{99.5} of 1536 μg/mL. Importantly, the same isolates were highly susceptible to H2B10B11 with a median MIC of 2 μg/mL H2B10B11 and median MBC_{99.5} of 8 μg/mL. Note that MIC values of H2B10B11 were determined after 72 h incubation, which is different to the MICs determined above for PM-477 (H2B10).

### Example 2: Distinct Killing of Gardnerella Cells in Biofilms by MDZ, CLI and PM-477 (H2B10)

Gardnerella dominated biofilms covering the human vaginal epithelial cells are now generally considered as a hallmark of BV. Thus, it was tested how effective antibiotics and PM-477 (H2B10) are in penetrating and killing in vitro pre-formed, 40-72 h old biofilms of various Gardnerella strains. MBEC values of ancestor (non-passaged) Gardnerella isolates are shown in Table 3 below. Interestingly, Gardnerella strains, which are all highly susceptible to CLI in suspension (MICs ranging from 0.01 to 1 μg/mL), become tolerant to CLI when grown as biofilms (Table 3 and FIG. 1), with an MBEC of up to ≥512 μg/mL. MDZ removed the biofilms beyond LOD at MBECs ranging from 8-128 μg/mL, while the endolysin PM-477 (H2B10) was able to kill all six Gardnerella strains grown as biofilm at MBECs lower than any of the antibiotics, i.e., <2-32 μg/mL. The reduction in biofilm CFU of a representative Gardnerella strain (G. vaginalis ATCC14018^{T}) by MDZ, CLI, and PM-477 (H2B10) is depicted in FIG. 1.

TABLE 3

| | MBEC [μg/mL] | | |
|---|---|---|---|
| | | PM-477 | |
| Ancestor Gardnerella isolate | MDZ | (H2B10) | CLI |
| G. vaginalis ATCC14018 | 32 | 32 | >512 |
| G. vaginalis UGent 09.07 | 32 | 16 | >512 |
| G. vaginalis UGent BV50 | 128 | 2 | >512 |
| G. vaginalis UGent BV111.5 | 32-128 | 8-32 | >512 |
| G. vaginalis UGent FB049 | 2-8 | 0.5-2 | >512 |
| G. swidsinskii GS 9838-1 | 32 | <2 | >512 |
| G. swidsinskii UGent BV 7.1 | 32 | 32 | <4 |
| G. piotii UGent 18.01 | 8 | 8 | <4 |
| G. piotii UGent 21.28 | 8-32 | 2-8 | >512 |

### Example 3: Gardnerella spp. That are Initially Susceptible to MDZ Quickly Become Resistant Upon Serial Passaging Rounds

Six Gardnerella strains which were initially either susceptible to MDZ (G. vaginalis ATCC14018^{T}, MIC 8 μg/mL) or at least to the more potent MDZ hydroxy metabolite (MDZ-OH, MIC between 2-16 μg/mL, data not shown) were serially passaged in MDZ or MDZ-OH at sub-MIC concentrations, so that growth was impaired but not completely inhibited. Upon passaging, the MIC for both MDZ and the MDZ-OH metabolite increased strongly, and all Gardnerella strains reached the resistance breakpoint of ≥32 μg/mL within 5 rounds of passaging (see FIG. 2). After 9 rounds of passaging, 4 out of 6 strains could no longer be inhibited even by the maximal concentration in use (MIC>512 μg/mL) (FIG. 2). In parallel, one representative of G. vaginalis (ATCC 14018^{T}) was passaged 25 times with PM-477 (H2B10). The MIC of PM-477 (H2B10) increased only slightly over the 25 rounds of passaging, to 8 μg/mL (FIG. 2).

### Example 4: Passaged Isolates with Acquired MDZ Resistance Remain Susceptible to PM-477 (H2B10)

Next, it was tested to what extent the resistance of Gardnerella cells toward MDZ also impacts the susceptibility to the endolysin PM-477 (H2B10). G. vaginalis (ATCC 14018) passaged for 25-days on MDZ was exposed to different concentrations of MDZ and PM-477 (H2B10) for 1, 5, and 24 hours and then the bactericidal effect was assessed by quantitative plating on Choc agar plates. The susceptibility of Gardnerella strains before and after 8 to 9-times passaging with sub-MIC concentrations of MZD or MDZ-OH is shown in Table 4 below. All MICs determined after 48 h of incubation.

TABLE 4

| | | MIC [μg/mL] | | | MIC [μg/mL] | | | MBEC [μg/mL] | |
|---|---|---|---|---|---|---|---|---|---|
| Gardnerella | Passaging | before passaging | | | passaged | | | passaged | |
| Strains | Antibiotic | MDZ | MDZ-OH | PM-477 | MDZ | MDZ-OH | PM-477 | MDZ | PM-477 |
| G. vaginalis | MDZ | 8 | 2 | 0.06 | >512 | >512 | 0.13 | >2048 | 2-8 |
| (ATCC 14018^{T}) | | | | | | | | | |
| G. vaginalis | MDZ-OH | 64 | 8 | 0.25 | >2048 | >512 | 0.25 | >2048 | 2-4 |
| (UGent 09.07)¹ | | | | | | | | | |
| G. vaginalis | MDZ-OH | 32 | 4 | 0.5 | >2048 | >512 | 0.25 | >2048 | 1-8 |
| (UGent BV50) | | | | | | | | | |
| G. swidsinskii | MDZ-OH | 256 | 16 | 0.03 | >512 | >512 | 0.06 | >2048 | 2 |
| (GS 9838-1^{T}) | | | | | | | | | |
| G. swidsinskii | MDZ-OH | 256 | 32 | 0.25 | >2048 | >512 | 0.06 | >2048 | 2 |
| (UGent BV 7.1) | | | | | | | | | |
| G. piotii | MDZ-OH | 32 | 8 | 1 | >2048 | >512 | 2 | >2048 | <1 |
| (UGent 18.01^{T}) | | | | | | | | | |

¹G. vaginalis (UGent09.07) was only passaged 8 times with MDZ-OH.

For the ancestral strain, there is a clear dose- and time-dependency of viability upon exposure to MDZ (FIG. 3A). In contrast, the G. vaginalis strain passaged on MDZ can tolerate very high concentrations of MDZ up to 2 mg/ml for 1 and 5 hours without any loss in viability, and the treatment with 2 mg/ml for 24 h resulted in only a 2-log reduction compared to the buffer treated control. When G. vaginalis (ATCC 14018) was exposed to PM-477 (H2B10), the susceptibility was similar for the ancestral and the passaged strains (FIG. 3B). After 1 h of treatment, 10 μg/mL PM-477 (H2B10) reduced a suspension of 108 CFU/mL by 3.0 and 2.8 log units for the ancestral and passaged strain, respectively. Also, the MIC values of PM-477 (H2B10) were very similar for ancestral and MDZ-passaged strains (Table 4). This indicates that the acquired resistance against MDZ does not interfere with the mode of action of the endolysin.

All MDZ or MDZ-OH passaged Gardnerella strains were still capable of forming biofilms similar in thickness and CFU-count as their respective non-passaged ancestors (data not shown). The MBEC of MDZ on Gardnerella strains passaged on MDZ or MDZ-OH was >2048 μg/mL for all six passaged strains (FIG. 4A). Of these, only one G. vaginalis strains (ATCC 14018) initially had a MIC below the resistance breakpoint. However, given that intravaginal MDZ (typically a 0.75% crème, i.e., 7.5 mg/mL) can establish concentrations in the mg/mL range in vaginal fluid, strains with MICs above the EUCAST breakpoint were also passaged and tested (pre-passaging MIC range 8-256 μg/mL, see Table 4).

In contrast, all strains were highly susceptible to PM-477 (H2B10), with MBECs in the range of 1-8 μg/ml (FIG. 4B, Table 3).

### Example 5: Gardnerella BV Patient Isolates of Various Origins are Susceptible to H2B10B11

MIC and MBC_{99.5} of H2B10B11 on a panel of about 100 Gardnerella type strains and patient isolates from various geographic origins were determined following the CLSI protocol. Since most of the clinical isolates were poorly growing strains, the MIC reaction plates were incubated for 72 h. For a subset of fast-growing strains thereof and for some additional strains, MIC determination was performed after 48 h of incubation to bridge the results to earlier assessed MIC values (FIG. 6). In total, MIC was determined for 104 different strains (the MIC of 15 strains was determined both, after 48 h and 72 h). Both MIC and MBC_{99.5} values were one concentration lower when the readout was done after 48 h compared to 72 h. The strains summarized in “All Gardnerella (72 h)” were sub-grouped according to their geographical origin or to their species (note that 2 isolates could not be identified to the species level, which is why they do not appear in the species subgroups). The number of strains in each group is stated on the right site of the graph. Importantly, there was no significant difference between the susceptibility of isolates from African and European BV patients (see Materials and Methods section for source of isolates) to H2B10B11 (unpaired t-test of log2-transformed data, p=0.1221). Likewise, there was no significant difference between the MICs of G. vaginalis and G. leopoldii/swidsinskii (unpaired t-test of log2-transformed data, p=0.5076).

1. A recombinant Gardnerella-specific endolysin for use in treating bacterial vaginosis, wherein the endolysin is to be administered to a patient who previously failed a treatment with antibiotics and/or who suffers from bacterial vaginosis wherein the infective bacteria are resistant to a treatment with antibiotics, in particular wherein said antibiotics are nitroimidazole and/or Clindamycin.

2. The endolysin for use according to claim 1, wherein said treatment with antibiotics is a treatment with Metronidazole, Tinidazole, Secnidazole, Clindamycin or any combination thereof.

3. The endolysin for use according to claim 1 or 2, wherein said patient suffers from recurrent bacterial vaginosis, preferably wherein said patient had two or more episodes of BV in 6 months or had three or more episodes of BV in 12 months.

4. The endolysin for use according to any one of the preceding claims, wherein said bacterial vaginosis is characterized by the presence of infective bacteria of the species Gardnerella vaginalis sensu stricto, Gardnerella leopoldii, Gardnerella piotii, Gardnerella swidsinskii, and/or any other species in the genus Gardnerella.

5. The endolysin for use according to any one of the preceding claims, wherein said endolysin has killing activity against species in the genus Gardnerella.

6. The endolysin for use according to any one of the preceding claims, wherein said endolysin has killing activity against Gardnerella vaginalis sensu stricto, Gardnerella leopoldii, Gardnerella piotii, and/or Gardnerella swidsinskii.

7. The endolysin for use according to any one of the preceding claims, wherein said endolysin has no killing activity against Lactobacilli crispatus, Lactobacilli gasseri, and/or Lactobacilli jensenii.

8. The endolysin for use according to any one of the preceding claims, wherein said endolysin comprises or consists of
(i) a N-terminal catalytic domain, or a functional variant thereof;
(ii) a C-terminal cell-wall binding region, or a functional variant thereof, wherein the C-terminal cell-wall binding region comprises or consists of at least one cell-wall binding domain; and
(iii) optionally a linker region between the N-terminal catalytic domain and the C-terminal cell-wall binding region.

9. The endolysin for use according to claim 8, wherein the catalytic domain is a polypeptide comprising or consisting of the amino acid sequence of any one of SEQ ID NOs: 2 to 10 or any functional variant thereof having at least 80% identity with the amino acid sequence of any one of SEQ ID NOs: 2 to 10, preferably a polypeptide comprising the amino acid sequence of SEQ ID NO: 3.

10. The endolysin for use according to claim 8 or claim 9, wherein the cell-wall binding domain is a polypeptide comprising or consisting of the amino acid sequence of any one of SEQ ID NOs: 11 to 28, or any functional variant thereof having at least 80% identity with the amino acid sequence of any one of SEQ ID NOs: 11 to 28.

11. The endolysin for use according to any one of claims 8 to 10, wherein the C-terminal cell-wall binding region comprises or consists of a first cell-wall binding domain and a second cell-wall binding domain,
wherein said first cell-wall binding domain is a polypeptide comprising or consisting of the amino acid sequence of SEQ ID NO: 23, or any functional variant thereof having at least 80% identity with the amino acid sequence of SEQ ID NO: 23, and
wherein said second cell-wall binding domain is a polypeptide comprising or consisting of the amino acid sequence of SEQ ID NO: 24 or SEQ ID NO: 26, or any functional variant thereof having at least 80% identity with the amino acid sequence of SEQ ID NO: 24 or SEQ ID NO: 26.

12. The endolysin for use according to any one of claims 8 to 11, whereby the endolysin is functional, wherein the function comprises the ability to lyse the cell wall of Gardnerella.

13. The endolysin for use according to any one of the preceding claims, wherein said endolysin is a polypeptide having at least 80% sequence identity with the amino acid sequence as provided in SEQ ID NO: 1 and having a killing activity against Gardnerella.

14. The endolysin for use according to any one of the preceding claims, wherein said endolysin comprises or consists of
(i) a N-terminal catalytic domain consisting of a polypeptide which comprises or consists of the amino acid sequence of SEQ ID NO: 3; and
(ii) a C-terminal cell-wall binding region comprising or consisting of a first cell-wall binding domain and a second cell-wall binding domain,
wherein said first cell-wall binding is a polypeptide comprising or consisting of the amino acid sequence of SEQ ID NO: 23, and said second cell-wall binding domain is a polypeptide comprising or consisting of the amino acid sequence of SEQ ID NO: 24 or SEQ ID NO: 26.

15. The endolysin for use according to claim 11 or claim 14, wherein said first cell-wall binding domain is located N-terminally of said second cell-wall binding domain.

16. The endolysin for use according to any one of the preceding claims, wherein said endolysin comprises or consists of the amino acid sequence as provided in SEQ ID NO: 1 or SEQ ID NO: 37.

17. The endolysin for use according to any one of the preceding claims, which is to be administered locally into the vagina of a female subject and/or into or on the glans penis, prepuce or urethral entry of a male subject.

18. The endolysin for use according to any one of the preceding claims, which is to be co-administered with a compound or composition which adjust the pH of the vagina to 4.0-6.0.

19. A pharmaceutical composition comprising a recombinant Gardnerella-specific endolysin and optionally a pharmaceutically acceptable carrier and/or diluent for use in treating bacterial vaginosis in a patient who previously failed a treatment with antibiotics and/or who suffers from bacterial vaginosis wherein the infective bacteria are resistant to a treatment with antibiotics.

20. The pharmaceutical composition for use according to claim 19, wherein said treatment with antibiotics is a treatment with Metronidazole, Tinidazole, Secnidazole, Clindamycin or any combination thereof.

21. The pharmaceutical composition for use according to claim 19 or claim 20, wherein said patient suffers from recurrent bacterial vaginosis, preferably wherein said patient had two or more episodes of BV in 6 months or had three or more episodes of BV in 12 months.

22. The pharmaceutical composition for use according to any one of claims 19 to 21, wherein said bacterial vaginosis is characterized by the presence of infective bacteria of the species Gardnerella vaginalis sensu stricto, Gardnerella leopoldii, Gardnerella piotii, Gardnerella swidsinskii, and/or any other species in the genus Gardnerella.

23. The pharmaceutical composition for use according to any one of claims 19 to 22, wherein said endolysin has killing activity against species in the genus Gardnerella.

24. The pharmaceutical composition for use according to any one of claims 19 to 23, wherein said endolysin has killing activity against Gardnerella vaginalis sensu stricto, Gardnerella leopoldii, Gardnerella piotii, and/or Gardnerella swidsinskii.

25. The pharmaceutical composition for use according to any one of claims 19 to 24, wherein said endolysin has no killing activity against Lactobacilli crispatus, Lactobacilli gasseri, and/or Lactobacilli jensenii.

26. The pharmaceutical composition for use according to any one of claims 19 to 26, wherein said endolysin comprises or consists of
(i) a N-terminal catalytic domain, or a functional variant thereof;
(ii) a C-terminal cell-wall binding region, or a functional variant thereof, wherein the C-terminal cell-wall binding region comprises or consists of at least one cell-wall binding domain; and
(iii) optionally a linker region between the N-terminal catalytic domain and the C-terminal cell-wall binding region.

27. The pharmaceutical composition for use according to claim 26, wherein the catalytic domain is a polypeptide comprising or consisting of the amino acid sequence of any one of SEQ ID NOs: 2 to 10 or any functional variant thereof having at least 80% identity with the amino acid sequence of any one of SEQ ID NOs: 2 to 10, preferably a polypeptide comprising the amino acid sequence of SEQ ID NO: 3.

28. The pharmaceutical composition for use according to claim 26 or claim 27, wherein the cell-wall binding domain is a polypeptide comprising or consisting of the amino acid sequence of any one of SEQ ID NOS: 11 to 28, or any functional variant thereof having at least 80% identity with the amino acid sequence of any one of SEQ ID NOS: 11 to 28.

29. The pharmaceutical composition for use according to any one of claims 26 to 28, wherein the C-terminal cell-wall binding region comprises or consists of a first cell-wall binding domain and a second cell-wall binding domain,
wherein said first cell-wall binding domain is a polypeptide comprising or consisting of the amino acid sequence of SEQ ID NO: 23, or any functional variant thereof having at least 80% identity with the amino acid sequence of SEQ ID NO: 23, and
wherein said second cell-wall binding domain is a polypeptide comprising or consisting of the amino acid sequence of SEQ ID NO: 24 or SEQ ID NO: 26, or any functional variant thereof having at least 80% identity with the amino acid sequence of SEQ ID NO: 24 or SEQ ID NO: 26.

30. The pharmaceutical composition for use according to any one of claims 26 to 29, whereby the endolysin is functional, wherein the function comprises the ability to lyse the cell wall of Gardnerella.

31. The pharmaceutical composition for use according to any one of claims 19 to 30, wherein said endolysin is a polypeptide having at least 80% sequence identity with the amino acid sequence as provided in SEQ ID NO: 1 and having a killing activity against Gardnerella.

32. The pharmaceutical composition for use according to any one of claims 19 to 31, wherein said endolysin comprises or consists of
(i) a N-terminal catalytic domain consisting of a polypeptide which comprises or consists of the amino acid sequence of SEQ ID NO: 3; and
(ii) a C-terminal cell-wall binding region comprising or consisting of a first cell-wall binding domain and a second cell-wall binding domain,
wherein said first cell-wall binding domain is a polypeptide comprising or consisting of the amino acid sequence of SEQ ID NO: 23, and said second cell-wall binding domain is a polypeptide comprising or consisting of the amino acid sequence of SEQ ID NO: 24 or SEQ ID NO: 26.

33. The pharmaceutical composition for use according to claim 29 or claim 32, wherein said first cell-wall binding domain is located N-terminally of said second cell-wall binding domain.

34. The pharmaceutical composition for use according to any one of claims 19 to 33, wherein said endolysin comprises or consists of the amino acid sequence as provided in SEQ ID NO: 1 or SEQ ID NO: 37.

35. The pharmaceutical composition for use according to any one of claims 19 to 34, which is to be administered locally into the vagina of a female subject and/or into or on the glans penis, prepuce or urethral entry of a male subject.

36. The pharmaceutical composition for use according to any one of claims 19 to 35, wherein the endolysin is to be co-administered with a compound or composition which adjust the pH of the vagina to 4.0-6.0.

37. The pharmaceutical composition for use according to any one of claims 19 to 35, which further comprises a compound or composition which adjust the pH of the vagina to 4.0-6.0.

38. A method of treating bacterial vaginosis in a patient who previously failed a treatment with antibiotics and/or who suffers from bacterial vaginosis wherein the infective bacteria are resistant to a treatment with antibiotics, wherein the method comprises administering to said patient a therapeutically effective amount of a recombinant Gardnerella-specific endolysin or a pharmaceutical composition comprising a recombinant Gardnerella-specific endolysin.

39. The method according to claim 38, wherein said treatment with antibiotics is a treatment with Metronidazole, Tinidazole, Secnidazole, Clindamycin or any combination thereof.

40. The method according to claim 38 or claim 39, wherein said patient suffers from recurrent bacterial vaginosis, preferably wherein said patient had two or more episodes of BV in 6 months or had three or more episodes of BV in 12 months.

41. The method according to any one of claims 38 to 40, wherein said bacterial vaginosis is characterized by the presence of infective bacteria of the species Gardnerella vaginalis sensu stricto, Gardnerella leopoldii, Gardnerella piotii, Gardnerella swidsinskii, and/or any other species in the genus Gardnerella.

42. The method according to any one of claims 38 to 41, wherein said endolysin has killing activity against species in the genus Gardnerella.

43. The method according to any one of claims 38 to 42, wherein said endolysin has killing activity against Gardnerella vaginalis sensu stricto, Gardnerella leopoldii, Gardnerella piotii, and/or Gardnerella swidsinskii.

44. The method according to any one of claims 38 to 43, wherein said endolysin has no killing activity against Lactobacilli crispatus, Lactobacilli gasseri, and/or Lactobacilli jensenii.

45. The method according to any one of claims 38 to 44, wherein said endolysin comprises or consists of
(i) a N-terminal catalytic domain, or a functional variant thereof;
(ii) a C-terminal cell-wall binding region, or a functional variant thereof, wherein the C-terminal cell-wall binding region comprises or consists of at least one cell-wall binding domain; and
(iii) optionally a linker region between the N-terminal catalytic domain and the C-terminal cell-wall binding region.

46. The method according to claim 45, wherein the catalytic domain is a polypeptide comprising or consisting of the amino acid sequence of any one of SEQ ID NOs: 2 to 10 or any functional variant thereof having at least 80% identity with the amino acid sequence of any one of SEQ ID NOs: 2 to 10, preferably a polypeptide comprising the amino acid sequence of SEQ ID NO: 3.

47. The method according to claim 45 or claim 46, wherein the cell-wall binding domain is a polypeptide comprising or consisting of the amino acid sequence of any one of SEQ ID NOs: 11 to 28, or any functional variant thereof having at least 80% identity with the amino acid sequence of any one of SEQ ID NOs: 11 to 28.

48. The method according to any one of claims 45 to 47, wherein the C-terminal cell-wall binding region comprises or consists of a first cell-wall binding domain and a second cell-wall binding domain,
wherein said first cell-wall binding domain is a polypeptide comprising or consisting of the amino acid sequence of SEQ ID NO: 23, or any functional variant thereof having at least 80% identity with the amino acid sequence of SEQ ID NO: 23, and
wherein said second cell-wall binding domain is a polypeptide comprising or consisting of the amino acid sequence of SEQ ID NO: 24 or SEQ ID NO: 26, or any functional variant thereof having at least 80% identity with the amino acid sequence of SEQ ID NO: 24 or SEQ ID NO: 26.

49. The method according to any one of claims 45 to 48, whereby the endolysin is functional, wherein the function comprises the ability to lyse the cell wall of Gardnerella.

50. The method according to any one of claims 38 to 49, wherein said endolysin is a polypeptide having at least 80% sequence identity with the amino acid sequence as provided in SEQ ID NO: 1 and having a killing activity against Gardnerella.

51. The method for use according to any one of claims 38 to 50, wherein said endolysin comprises or consists of
(i) a N-terminal catalytic domain consisting of a polypeptide which comprises or consists of the amino acid sequence of SEQ ID NO: 3; and
(ii) a C-terminal cell-wall binding region comprising or consisting of a first cell-wall binding domain and a second cell-wall binding domain,
wherein said first cell-wall binding domain is a polypeptide comprising or consisting of the amino acid sequence of SEQ ID NO: 23, and said second cell-wall binding domain is a polypeptide comprising or consisting of the amino acid sequence of SEQ ID NO: 24 or SEQ ID NO: 26.

52. The method according to claim 48 or claim 51, wherein said first cell-wall binding domain is located N-terminally of said second cell-wall binding domain.

53. The method according to any one of claims 38 to 52, wherein said endolysin comprises or consists of the amino acid sequence as provided in SEQ ID NO: 1 or SEQ ID NO: 37.

54. The method according to any one of claims 38 to 53, wherein the endolysin or composition is to be administered locally into the vagina of a female subject and/or into or on the gians penis, prepuce or urethral entry of a male subject.

55. The method according to any one of claims 38 to 54, wherein the endolysin or composition is to be co-administered with a compound or composition which adjust the pH of the vegina to 4.0-6.0.


SEQUENCE LISTING (37 sequences)

SEQ ID NO: 1 - protein, 305 aa, synthetic construct
  source 1..305: note = H2B10
SKRGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITGFCNKLESCGYYAGFYTSLSTANNLVSAHVRNRYALWIAQWNTHCSYQGSYGLWQYSSSGSVPGVAGRVDMDYAYVDYPSIIKNVGLNGYKNGGSYTAPQISSIDEVAREVINGDWGNGNERKQRLTSAGYDYASVQNKVNELLGVKAYRKSVDELAREVIRGTWGNGSTRKQRLTQAGYDYNAVQKRVNELL

SEQ ID NO: 2 - protein, 196 aa, synthetic construct
  source 1..196: note = H1
MSKKGIDVSEWQGDIDFNAVKASGVEFVIIRAGYGIGCKDKWFEQNYRKAKTAGLDVGAYWYSYANSGFEAAEEAQSLMNMLSGKSFEYPVYFDLEEKSQLNRGRAFCDSLITSFCNKLEACGYYAGFYTSLSTANNLVSAHVRNRYALWIAQWNTHCNYQGSYGLWQYSSNGSVPGVAGRVDMDYAYVDYPSIIK

SEQ ID NO: 3 - protein, 196 aa, synthetic construct
  source 1..196: note = H2
MSKRGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITGFCNKLESCGYYAGFYTSLSTANNLVSAHVRNRYALWIAQWNTHCSYQGSYGLWQYSSSGSVPGVAGRVDMDYAYVDYPSIIK

SEQ ID NO: 4 - protein, 196 aa, synthetic construct
  source 1..196: note = H3
MSKKGIDVSVWQGDIDFNSVKASGVEFVIIRAGYGIGHKDKWFEENYRKAKTAGLDVGSYWYSYASSAGEASEEAQSCVNILSGKSFEYPIYFDLEEKSQLNRGRDFCDSLITSFCNKLEACGYYAGFYTSLSVANNLVSSHVRDRYALWIAQWNTHCSYQGSYGLWQYSSSGSVNGIAGRVDMDYAYVDYPSVIK

SEQ ID NO: 5 - protein, 196 aa, synthetic construct
  source 1..196: note = H4
MSKKGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITSFCSKLETYGYYAGFYTSLSVVNNLVSAHVRDRYALWIAQWNTHCSYQGSYGLWQYSSSGSVPGVAGRVDMDYAYVDYPSIIK

SEQ ID NO: 6 - protein, 196 aa, synthetic construct
  source 1..196: note = H5
MSKKGIDVSEWQGDIDFNAVKASGVEFVIIRAGYGIGCKDKWFEQNYRKAKTAGLDVGAYWYSYANSSSEAAEEAQSCANMLSGKSFEYPVYFDLEEKSQLNRGRAFCDSLITGFCSKLEACGYYAGFYTSLSTANNLVSAHVRNRYALWIAQWNTHCSYQGSYGLWQYSSNGSVPGVAGRVDMDYAYKDYPSIIK

SEQ ID NO: 7 - protein, 196 aa, synthetic construct
  source 1..196: note = H7
MSKKGIDVSEWQGDIDFNAVKASGVEFVIIRAGYGIGCKDKWFEQNYRKAKTAGLDVGAYWYSYANSASEAAEEAQSCANMLSGKSFEYPVYFDLEEKSQLNRGRAFCDSLITSFCSKLETYGYYAGFYTSLSTANNLVSSHVRNRYALWIAQWNTHCSYQGSYGLWQYSSSGSVPGVAGRVDMDYAYKDYPSIIK

SEQ ID NO: 8 - protein, 196 aa, synthetic construct
  source 1..196: note = H10
MSKRGIDVSVWQGDIDFNAVKASGVEFVIIRAGYGIGHKDKWFEENYRKAKTVGLDVGAYWYSYASSAGEASEEAQSCVNILSGKSFEYPVYFDLEEKSQLNRGRDFCDSLITSFCNKLEACGYYAGFYTSLSVANNLVSSHVRDRYALWIAQWNTHCSYQGSYGLWQYSSSGSVNGIAGRVDMDYAYVDYPSVIK

SEQ ID NO: 9 - protein, 196 aa, synthetic construct
  source 1..196: note = H11
MSKRGIDVSVWQGDIDFNAVKASGVEFVIIRAGYGIGHKDKWFEQNYRKAKTTGLDVGAYWYSYASSAGEAAEEAQSCVNILSGKSFEYPVYFDLEEKSQLNRGRDFCDSLITSFCNKLETYGYYAGFYTSLSVANNLVSSHVRDRYALWIAQWNTHCDYQGSYGLWQYSSSGSVDGIAGRVDMDYTYVDYPSVIK

SEQ ID NO: 10 - protein, 196 aa, synthetic construct
  source 1..196: note = H12
MSKKGIDVSVWQGDIDFNAVKASGVEFVIIRAGYGIGHKDKWFEENYRKAKTAGLDVGSYWYSYASSAGEVALEAQSCVNILSGKSFEYPVYFDLEEKSQLNRGRDFCDSLITSFCNKLEACGYYAGFYTSLSVANNLVSSHVRDRYALWIAQWNTHCSYQGSYGLWQYSSSGSVNGIAGRVDMDYAYVDYPSVIK

SEQ ID NO: 11 - protein, 49 aa, synthetic construct
  source 1..49: note = B1_N
DQAARTSSIDEVAREVINGAWGNGSTRKQRLTSAGYDYASVQNKVNELL

SEQ ID NO: 12 - protein, 49 aa, synthetic construct
  source 1..49: note = B1_C
GVKACRKSVDELAREVIRGAWGNGSTRKQRLAQAGYDYDTVQKRVNELL

SEQ ID NO: 13 - protein, 49 aa, synthetic construct
  source 1..49: note = B2_N
DQAARTSSIDEVAREVINGAWGNGNERKQRLTSAGYDYASVQNKVNELL

SEQ ID NO: 14 - protein, 49 aa, synthetic construct
  source 1..49: note = B2_C
GVKACRKSVDEIAREVIRGTWGNGSTRKQRLTQAGYDYDTVQKRVNELL

SEQ ID NO: 15 - protein, 49 aa, synthetic construct
  source 1..49: note = B3_N
YTAPQTSSIDEVAREVINGDWGNGNDRKNRLISAGYDYASVQNKVNELL

SEQ ID NO: 16 - protein, 49 aa, synthetic construct
  source 1..49: note = B3_C
GVKAYRKSVDELAREVIRGTWGNGSMRKHRLTQAGYDYDAVQKRVNELL

SEQ ID NO: 17 - protein, 49 aa, synthetic construct
  source 1..49: note = B4_N
DQATRTSSIDEVAREVINGAWGNGNERKQRLTSAGYDYASVQNKVNKLL

SEQ ID NO: 18 - protein, 49 aa, synthetic construct
  source 1..49: note = B4_C
GVKAYRKSVDELAREVIRGTWGNGNERKQRLAQAGYDYDTVQKRVNELL

SEQ ID NO: 19 - protein, 49 aa, synthetic construct
  source 1..49: note = B5_N
DQAARTSSIDEVAREVINGAWGNGNERKQRLTQAGYDYTSVQNKVNKLL

SEQ ID NO: 20 - protein, 49 aa, synthetic construct
  source 1..49: note = B5_C
GVKACRKSVDELAREVIRGTWGNGNERKNRLTQAGYDYDTVQKRVNELL

SEQ ID NO: 21 - protein, 49 aa, synthetic construct; identical to SEQ ID NO: 19
  source 1..49: note = B7_N

SEQ ID NO: 22 - protein, 49 aa, synthetic construct; identical to SEQ ID NO: 20
  source 1..49: note = B7_C

SEQ ID NO: 23 - protein, 49 aa, synthetic construct
  source 1..49: note = B10_N
YTAPQISSIDEVAREVINGDWGNGNERKQRLTSAGYDYASVQNKVNELL

SEQ ID NO: 24 - protein, 49 aa, synthetic construct
  source 1..49: note = B10_C
GVKAYRKSVDELAREVIRGTWGNGSTRKQRLTQAGYDYNAVQKRVNELL

SEQ ID NO: 25 - protein, 49 aa, synthetic construct
  source 1..49: note = B11_N
YTAPQTSSIDEVAREVINGDWGNGNERKNRLTSAGYDYTSVQNKVNELL

SEQ ID NO: 26 - protein, 49 aa, synthetic construct
  source 1..49: note = B11_C
GVKAYRKSVDELAREVIRGTWGNGSTRKQRLTQAGYDYDAVQKRVNELL

SEQ ID NO: 27 - protein, 49 aa, synthetic construct
  source 1..49: note = B12_N
YTAPQTSSIDEVAREVINGDWGNGIERKNRLTSAGYDYTSVQNKVNELL

SEQ ID NO: 28 - protein, 49 aa, synthetic construct
  source 1..49: note = B12_C
GVKAYRKSVDELAREVIRGTWGNGKTRKQRLTQAGYDYNAVQKRVNELL

SEQ ID NO: 29 - protein, 12 aa, synthetic construct
  source 1..12: note = L1
NAGLNGCKNGGS

SEQ ID NO: 30 - protein, 12 aa, synthetic construct
  source 1..12: note = L2
NVGLNGCKNGGS

SEQ ID NO: 31 - protein, 12 aa, synthetic construct
  source 1..12: note = L3, L12
NAGLNGYQNGGS

SEQ ID NO: 32 - protein, 12 aa, synthetic construct
  source 1..12: note = L4
NVGLNGCKNGGS

SEQ ID NO: 33 - protein, 12 aa, synthetic construct
  source 1..12: note = L5, L6, L7
NAGLNGCKNGGS

SEQ ID NO: 34 - protein, 12 aa, synthetic construct
  source 1..12: note = L10
NAGLNGYKNGGS

SEQ ID NO: 35 - protein, 12 aa, synthetic construct
  source 1..12: note = L11
KAGLNGYKNGGS

SEQ ID NO: 36 - protein, 12 aa, synthetic construct
  source 1..12: note = Lx
  VARIANT 1: note = X is N or K
  VARIANT 2: note = X is A or V
  VARIANT 7: note = X is Y or C
  VARIANT 8: note = X is K or Q
XXGLNGXXNGGS

SEQ ID NO: 37 - protein, 306 aa, synthetic construct
  source 1..306: note = H2B10B11
MSKRGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITGFCNKLESCGYYAGFYTSLSTANNLVSAHVRNRYALWIAQWNTHCSYQGSYGLWQYSSSGSVPGVAGRVDMDYAYVDYPSIIKNVGLNGYKNGGSYTAPQISSIDEVAREVINGDWGNGNERKQRLTSAGYDYASVQNKVNELLGVKAYRKSVDELAREVIRGTWGNGSTRKQRLTQAGYDYDAVQKRVNELL